(19) United States
(12) Patent Application Publication
(10) Pub. No.: US 2017/0100353 A1
(43) Pub. Date: Apr. 13, 2017

(54) COMPOSITIONS AND METHODS THAT TARGET OLFACTORY RECEPTORS FOR REGULATION OF BREATHING

(71) Applicant: The Board of Trustees of the Leland Stanford Junio, Stanford, CA (US)

(72) Inventors: Mark Alan Krasnow, Stanford, CA (US); Andy Ju-An Chang, Menlo Park, CA (US)

(21) Appl. No.: 15/290,380

(22) Filed: Oct. 11, 2016

Related U.S. Application Data
(60) Provisional application No. 62/240,375, filed on Oct. 12, 2015.

(51) Int. Cl. A61K 31/19

(52) U.S. Cl. CPC A61K 31/19

(57) ABSTRACT

The olfactory receptor Olfr78/OR51E2 is shown to be expressed in the carotid body and to control breathing responses regulated by acute hypoxia sensing. Activation of Olfr78/OR51E2 increases breathing, while inhibitors of the receptor can counteract this activity. A native agonist of Olfr78/OR51E2 is shown to be lactate.

### CROSS REFERENCE

This application claims benefit of U.S. Provisional Patent Application No. 62/240,375, filed Oct. 12, 2015, which application is incorporated herein by reference in its entirety.

### FEDERALLY SPONSORED RESEARCH AND DEVELOPMENT

This invention was made with Government support under contract HL089989 awarded by the National Institutes of Health. The Government has certain rights in the invention.

### BACKGROUND OF THE INVENTION

In mammals, a first line of defense to low oxygen conditions (hypoxia) is to increase breathing, as low levels of blood oxygen in humans can cause irreversible brain damage after only a few minutes. While the molecular mechanism for slower adaptations to hypoxia (HIF) is known, mechanisms for rapid oxygen sensing are currently not well understood.

Located at the bifurcation of the carotid artery in the neck, the mammalian carotid body (CB) is a chemosensory organ that senses decreases in arterial blood oxygen to increase breathing within seconds; in hypoxia, CB glomus cells stimulate afferent nerves that connect to brainstem respiratory centers. This response is activated during exercise, pregnancy, and acclimatization to high altitude, and CB dysfunction contributes to diseases such as sudden infant death syndrome (SIDS), sleep-disordered breathing, congestive heart failure, and hypertension. In addition to its role in physiological homeostasis, oxygen regulation of breathing has ties to emotional state. Dyspnea, or the distressful feeling of breathlessness, can be triggered by hypoxia, and panic disorder patients with prominent hyperventilation during attacks have hypersensitive breathing responses to hypoxia.

CB glomus cells are neuroendocrine cells that form chemical synapses with afferent nerves, acutely releasing ATP and acetylcholine in hypoxia to stimulate breathing. A consensus model of CB oxygen sensing is that hypoxia closes background K⁺ channels on glomus cells, leading to plasma membrane depolarization, calcium influx through voltage-gated Ca⁺² channels, and neurotransmitter release. However, the oxygen-sensing pathway upstream of these steps has remained elusive.

The identification of oxygen sensing receptors and pathways in the carotid body is of general and clinical interest. The present invention addresses this issue.

### PUBLICATIONS

Kumar, P. & Prabhakar, N. R. Peripheral chemoreceptors: function and plasticity of the carotid body. Compr Physiol 2, 141-219 (2012); Marina, N. et al. Brainstem hypoxia contributes to the development of hypertension in the spontaneously hypertensive rat. Hypertension 65, 775-783 (2015). van der Schier, R., Roozekrans, M., van Velzen, M., Dahan, A. & Niesters, M. Opioid-induced respiratory depression: reversal by non-opioid drugs. F1000Prime Rep 6, 79 (2014).

Fleischer, J., Breer, H. & Strotmann, J. Mammalian olfactory receptors. Front Cell Neurosci 3, 9 (2009). Weber, M., Pehl, U., Breer, H. & Strotmann, J. Olfactory receptor expressed in ganglia of the autonomic nervous system. J Neurosci Res 68, 176-184 (2002). Matsuura, S. Chemoreceptor properties of glomus tissue found in the carotid region of the cat. J Physiol 235, 57-73 (1973). Bozza, T. et al. Mapping of class I and class II odorant receptors to glomerular domains by two distinct types of olfactory sensory neurons in the mouse. Neuron 61, 220-233 (2009).

Buckler, K. J. & Turner, P. J. Oxygen sensitivity of mitochondrial function in rat arterial chemoreceptor cells. J Physiol 591, 3549-3563 (2013). Neuhaus, et al. (2009) Activation of an olfactory receptor inhibits proliferation of prostate cancer cells. J Biol Chem 284: 16218-16225.

### SUMMARY OF THE INVENTION

Compositions and methods are provided for modulating of hypoxia-regulated breathing in a mammal, through inhibiting or activating the chemosensory receptor Olfr78/OR51E2. It is shown herein that Olfr78/OR51E2 is expressed in the carotid body and controls breathing responses regulated by acute hypoxia sensing. Activation of Olfr78/OR51E2 increases breathing, while inhibitors of the receptor can counteract this activity. A native agonist of Olfr78/OR51E2 is shown to be lactate. Olfr78/OR51E2 is expressed in blood vessels of the heart and lung, which may regulate vasodilation. A related receptor of the same family (Olfr558) is also expressed in similar tissues and has a similar ligand binding profile.

In one embodiments of the invention, a method is provided for modulating carotid body mediated changes in breathing, the method comprising contacting cells of the carotid body in a subject with an effective of an agonist or antagonist of Olfr78/OR51E2. In some embodiments the ligand specifically binds to OR51E2. An effective dose of an agonist is sufficient to increase breathing of the subject, relative to the level of ventilation in the absence of the agonist. For example, ventilation may be increased by about 5%, by about 10%, by about 15%, by about 20%, by about 25%, by about 30%, by about 35%, by about 40%, by about 45%, by about 50%, by about 55%, by about 65%, by about 75%, by about 85%, by about 95%, by about 100%, or more, e.g. 2-fold, 3-fold, etc. Stimulation of Olfr78/OR51E2 can also increase blood pressure. An effective dose of an antagonist is sufficient to counteract dysregulated signaling from Olfr78/OR51E2 that results in undesirable hyperventilation.

In one embodiment, methods are providing for screening candidate agents for activity in modulation of Olfr78/OR51E2. In some embodiments the candidate agent is screened for activity against OR51E2. Candidate agents can me initially screened by contacting with the receptor, including cells expressing the receptor, and determining changes in membrane polarization, Ca²⁺ influx, neurotransmitter release, and the like. Agents that activate the receptor may be further screened for activity in regulating the ventilator response of an animal. Certain candidate agonists include, without limitation, lactate, proprionate, β-ionone, steroids, including 6-dehydrotestosterone, ADT, 1,4,6-androstatriene-17β-ol-3-one, short chain fatty acids, and derivatives and analogs thereof. Agents that inhibit the receptor may be detected through counteraction of activation. Candidate antagonists include α-ionone, anti-sense RNAs and RNAi specific for Olfr78/OR51E2, antibodies that specifically bind to Olfr78/OR51E2 without activating the receptor; and the like.

In some embodiments, the methods of the invention are utilized in regulating hypoxic responses in infants, including without limitation premature infants. Oxygen extremes in infancy, such as hypoxia from poor lung and neural development in premature infants or hyperoxia from excessive supplemental oxygen, have detrimental effects on breathing stability and the ability to mount ventilatory responses to hypoxia.

In some embodiments the methods of the invention are utilized in regulating ventilator responses during administration of volatile anesthetics, which impair ventilatory responses to hypoxia and carotid body oxygen sensing. Counteracting breathing depression associated with administration of opiates is also of interest. Stimulation of the carotid body by drugs increases ventilation in patients with respiratory depression induced by opiates, a class of drugs commonly used for pain management.

In some embodiments the methods of the invention are utilized in regulating ventilator responses in sleep disordered breathing.

In some embodiments the methods of the invention are utilized in regulating congestive heart failure, and hypertension ventilator responses, in which hyperstimulation of the carotid body results in undesirable hyperventilation and hypertension. In such embodiments, an antagonist, or inhibitor of Olfr78/OR51E2 may be administered at an effective dose and for a period of time sufficient to regulate ventilation.

For the methods of the invention, administration of an effective dose of an agent that regulates breathing through Olfr78/OR51E2 may be provided acutely, e.g. for a period of from about 10 minutes, from about 20 minutes, from about 30 minutes, from about one hour, from about 2 hours, from about 4 hours, from about 6 hours, from about 8 hours, from about 12 hours to about 24 hours, to about 18 hours, to about 16 hours, to about 14 hours, to about 12 hours, to about 10 hours, to about 8 hours, to about 6 hours, to about 4 hours, to about 1 hour. Alternatively, for certain conditions it may be desirable to provide an effective dose of an agent for extended periods of time, e.g. from about 1 day, about 2 days, about 3 days, about 4 days, about 5 days, about 1 week, about 2 weeks, about 3 weeks, about 4 weeks, or more.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. Olfr78 is expressed in carotid body glomus cells. (a) Expression of 26,728 genes in adult mouse carotid body (CB) and adrenal medulla (AM) by RNA sequencing. Log₂ values of number of aligned reads per 10⁷ aligned reads generated. (b) OR genes highly expressed in CB and/or AM. X, fold enrichment (CB/AM). (a) (b) n=3 cohorts of 10 animals each. Data as mean (a) or mean±standard error of the mean (s.e.m., b). *P<0.05, **P<0.01 by paired t test by cohort. c-l, Expression of Olfr78 knock-in reporter mouse. (c-f), X-gal staining (blue) detects taulacZ (β-galactosidase) reporter activity. (c) Adrenal gland showing adrenal medulla (AM). Reporter not expressed. (d) Carotid bifurcation (dorsal view, superior cervical ganglion (SCG) removed). Reporter expressed in CB (dashed circle) and sporadic blood vessels (arrowhead). (e, f) Transverse section of carotid bifurcation (e) and close-up (f). (g, h) Immunostaining of CB sections. Olfr78 reporter expression (GFP; green) co-localized with CB glomus cell marker (tyrosine hydroxylase, TH; red; g) but not endothelial cell marker (CD31; red; (h). TH is also expressed in nerve fibers and SCG. DAPI (blue), nuclei. (i-k) X-gal stained carotid bifurcations (ventral view). (i) CB (dashed circle) innervated by carotid sinus nerve (filled arrowhead), a branch of glossopharyngeal nerve (open arrowhead). (j) “Miniglomerulus” (MG; dashed circle) innervated by glossopharyngeal nerve (arrowhead). (k) Petrosal ganglion (arrow), nodose/jugular ganglia (arrowhead). Reporter not expressed. (I) Olfr78 reporter expression (X-gal staining) during CB development. Filled circles, robust expression; open circles, not detected. Bars, 500 μm (c-e, i-k) and 100 μm (f-h).

FIG. 2. Ventilatory responses of Olfr78 null mutants to hypoxia and hypercapnia. Respiratory rate (RR), tidal volume (TV), and minute ventilation (MV=RR*TV) by whole body plethysmography of unrestrained, unanesthetized Olfr78^{+/+} and Olfr78^{-/-} littermates exposed to hypoxia (a, b) or hypercapnia (c, d). (a, b) Respiratory rate in hypoxia (a) and hypoxic response (b) as percent change in hypoxia (10% O₂) versus control (21% O₂). n=9 (+/+), 8 (−/−) animals. (c, d) Respiratory rate in hypercapnia (c) and hypercapnic response (d) as percent change in hypercapnia (5% CO₂) versus control (0% CO₂). n=4 (+/+), 5 (−/−) animals. Data as mean±s.e.m. *P<0.05, **P<0.01, ***P<0.001 by paired t test (a, c) or unpaired t test (b, d).

FIG. 3. Olfr78 mediates carotid body oxygen sensing. (a, b) CB sections from Olfr78^{+/+} control (a) and Olfr78^{-/-} knockout allele (b) in which GFP-IRES-taulacZ replaces Olfr78 coding region. GFP (green), tyrosine hydroxylase (TH; red), and DAPI (blue). Mutant CB shows normal organization. (c) Quantification of CB TH-positive cells. n=8 (+/+), 14 (−/−) CBs. Data as mean±s.e.m. P=0.454 by unpaired t test. (d-g), Transmission electron micrographs of Olfr78^{+/+} (d, e) and Olfr78^{-/-} (f, g) CBs. (e, g), close-ups of boxed regions. Both wild type and mutant glomus cells have large nuclei (asterisks), large dense core vesicles (open arrowheads), and small clear core vesicles (filled arrowheads). Bars, 100 μm (a, b), 600 nm (d, f), and 200 nm (e, g). (h, i), CB responses to hypoxia (h) and low pH (i) assayed by carotid sinus nerve discharge frequency (impulses/sec) of Olfr78^{+/+} and Olfr78^{+/-} controls (blue) and Olfr78^{-/-} mutants (red). (h), Hypoxia response as superperfusate changed from bubbling 95% O₂/5% CO₂ to 95% N₂/5% CO₂ (t=0 min) and back to 95% O₂/5% CO₂ (t=8 min, arrow). Gray line, representative time course of PO₂ in recording chamber. Discharge frequency of control nerves began increasing at PO₂=80 mmHg (t=6 min) and peaked at PO₂=60 mmHg (t=9 min). n=6 (3 +/+, 3 +/−), 5 (−/−) animals. i, Shift from pH 7.4 to pH 7.0. n=5 (+/+), 5 (−/−) animals. Data as mean±s.e.m. *P<0.05, **P<0.01, ***P<0.001 by unpaired t test.

FIG. 4. Lactate activates Olfr78 and carotid body sensory activity. (a), Lactate activation of Olfr78 expressed in HEK293T cells detected by dual reporter assay (see Methods). Cells transfected with pCl (empty vector, gray) or pCl-Rho-Olfr78 (epitope-tagged Olfr78, black). n=12 (4 transfected wells per plate on 3 plates performed on separate days). ANOVA for pCl data, P=0.478. (b), Arterial blood lactate of anesthetized Olfr78^{+/+} and Olfr78^{-/-} littermates exposed to hypoxia for 3 min. n=4 (+/+, 21% O₂), 4 (+/+, 10% O₂), 4 (−/−, 21% O₂), 6 (−/−, 10% O₂) animals. (c, d), Calcium response of GCaMP3-expressing glomus cells exposed to hypoxia (PO₂=40-50 mmHg), lactate (30 mM), and cyanide (2 mM), in whole mount and slice. (c), Time course of stimuli. Every data point differs from previous point (P<0.001 by paired t test). (d), GCaMP3 fluorescence change (F₁-F₀)/F₀ in percent. n=42 (whole mount, all stimuli), 29 (slice, hypoxia and cyanide), 22 (slice, lactate) cells. All changes differ from pre-stimulus (P<0.001 by paired t test). (e), Olfr78^{+/+} and Olfr78^{-/-} CB response to 30 mM lactate for 10 min, assayed by carotid sinus nerve activity. n=5 (+/+), 5 (−/−) animals. (a-e), Data as mean±s.e.m. *P<0.05, **P<0.01 by unpaired t test. (f), Model of oxygen sensing by Olfr78. In normoxia, pyruvate is efficiently used in Krebs cycle, supplying electrons to mitochondrial electron transport chain (ETC) to produce ATP. In hypoxia, lack of oxygen as final electron acceptor slows ETC, causing pyruvate to accumulate. Pyruvate is converted to lactate, which is secreted and binds Olfr78 on CB glomus cells, increasing intracellular calcium and transmitter release to afferent nerves to stimulate breathing.

FIG. 5. Model of oxygen sensing by the carotid body and the mitochondrion. (a), Anatomy and blood supply of the carotid body (CB). CB is located bilaterally at bifurcation of carotid artery (CA) in the neck. Its location can be variable as well as the source of its blood supply, which can come from branches of nearby internal and external carotid, occipital, pharyngeal arteries. Blood flows through fenestrated capillaries close to clusters of Type I glomus cells and drains from CB into jugular vein (JV) on ventral side. (b), Cellular organization of CB. CB is composed of several cell types, including Type I glomus cells (red) that sense changes in blood oxygen and are organized in clusters, Type II sustentacular cells (blue) that resemble neuroglia and surround glomus cell clusters, carotid sinus nerve (CSN) fibers that innervate glomus cells, and endothelial (E) and smooth muscle cells (not shown) that form the tortuous vasculature. (c), Oxygen-sensing respiratory circuit. The primary chemoreceptor for blood oxygen is the carotid body. A decrease in PaO₂ of arterial blood from normoxia (100 mmHg) to hypoxia (<80 mmHg) stimulates glomus cells to signal the carotid sinus nerve, a branch of glossopharyngeal nerve (GN) with cell bodies in petrosal ganglion (PG). Axons of the GN terminate in nucleus tractus solitarius (NTS) in brainstem, a site of many converging afferent inputs. The signal from NTS is transmitted to ventral respiratory group (VRG) that includes preBötzinger complex, a region essential for respiratory rhythm generation. From VRG, neurons project to premotor and motor neurons that innervate respiratory muscles, such as diaphragm and intercostal muscles. In addition to carotid body, vagus nerve afferents can also contribute to respiratory behaviors under specialized conditions. The vagus nerve innervates heart and lung and oxygen-sensitive cells of aortic body. (d), A current model of acute oxygen sensing by carotid body. A decrease in PaO₂ in blood causes a decrease in O₂ concentration inside carotid body glomus cells. This causes a decrease in activity of mitochondrial electron transport chain (ETC) and changes in other putative oxygen-sensing pathways, such as oxygen-sensitive K⁺ channels, heme oxygenase, AMP kinase, and hydrogen sulfide signaling. These changes are hypothesized to converge on oxygen-sensitive K⁺ channels, which close in hypoxia and depolarize the plasma membrane. Depolarization then opens voltage-gated Ca⁺² channels, leading to an increase in intracellular calcium that stimulates transmitter release to carotid sinus nerve to increase breathing. Mitochondria of carotid body cells are highly sensitive to hypoxia compared to other tissues, as assayed by imaging of mitochondrial membrane potential, NADH levels, and spectral properties. Drugs and mutations that inhibit the ETC mimic the effect of hypoxia on carotid body activity and breathing. (e), Regulation of lactate production by oxygen. In normoxia, pyruvate produced by glycolysis is transported into mitochondria and efficiently used in Krebs cycle to supply electrons to ETC to produce ATP. In hypoxia, lack of oxygen to act as the final electron acceptor limits electron transport, causing pyruvate to build up and become converted to lactate. The ETC poison cyanide inhibits the heme a3 subunit of cytochrome c oxidase to prevent transfer of electrons to oxygen, leading to lactate accumulation even in presence of adequate oxygen. Cytosolic lactate accumulation results in transport of lactic acid (lactate and H⁺) out of the cell by monocarboxylate transporters. In normoxia, lactate concentrations in blood, tissue, and tissue interstitium are 1-5 mM.

FIG. 6. RNA sequencing and whole genome microarrays detect Olfr78 transcripts enriched in the carotid body. (a), Histogram of frequency of genes for different levels of expression enrichment in carotid body (CB) relative to adrenal medulla (AM) by RNA sequencing. Log₂(CB/AM) values are shown, with data binned for every log₂ interval of 1.0 centered at integers. (b), Plot of log₂ values of reads per kilobase per million (RPKM) in CB and AM of all 1,126 olfactory receptor (OR) genes annotated in RefSeq shown in alphanumerical order. The five OR genes expressed at RPKM>2 (dashed line) are indicated. Samples that had no transcripts are plotted at a value of −7.1, just below the smallest RPKM value for ORs. Data presented in Table 1. (c), Comparison of expression levels of >34,000 genes in adult mouse CB and AM by whole genome microarrays. Plot shows log₂ of the ratio for CB relative to AM of the fluorescence intensity values for the 45,000 probe sets. The three probe sets for Olfr78 transcripts are indicated (circles). Expression of Olfr78 was significantly different between CB and AM for all three probe sets (P<0.05 by ANOVA with false discovery rate control). (d), Histogram of the frequency of genes for different levels of expression enrichment in CB relative to AM in microarray data. Log₂(CB/AM) values are shown, with data binned for every log₂ interval of 1.0 centered at integers. The three probe sets detecting Olfr78 mRNA (arrows) confirmed the RNA-seq data (a, FIGS. 1a, b, and Table 1) showing Olfr78 among the mRNAs most highly enriched in carotid body. Mouse CB Olfr78 expression is consistent with previous microarray data. (a-d), n=3 cohorts of 10 animals each. Data as mean. (e), Genomic locus showing the large cluster of ˜160 Class I OR genes on chromosome 7, with region encoding MOR18 subfamily (Olfr78, Olfr558, and Olfr557) expanded below. We did not detect transcripts in either tissue for Olfr557, which lies adjacent to Olfr558 in the cluster, or for the intervening (Olfr33, Olfr559) and intronic (Olfr560) ORs. Clusters of genes encoding globins, Trims, and USP proteins are also found with this OR cluster. Large box, coding sequence; arrowhead, coding orientation; small box, non-coding exons.

FIG. 7. Olfr78 and Olfr558 expression in tissues in the oxygen-sensing circuit. Expression of Olfr78 reporter in heterozygous (a) and homozygous (b-d) Olfr78-GFP-taulacZ reporter animals. a-c, Sections of carotid bifurcations stained for GFP (Olfr78 reporter; green), tyrosine hydroxylase (TH; red), and DAPI (nuclei; blue). (a), Section of CB showing co-expression of reporter GFP and TH in glomus cells. Monoallelic expression would predict that only half of TH-positive cells express the reporter. Arrowheads, clusters of glomus cells expressing both GFP and TH. (b, c), Sections of the same carotid bifurcation. Panels on right show close-ups of boxed region (petrosal ganglion, PG). No GFP-positive cells were found in petrosal ganglion. TH-positive nerve fibers (arrowheads) and cell bodies were found in glossopharyngeal nerve (GN) and petrosal ganglion. Dashed circle indicates vagus nerve (VN). NG/JG, nodose/jugular ganglia. (d), X-gal staining of a brain sagittal section. Reporter expression (blue) was restricted to olfactory bulb (arrowhead) in this section and complete brain serial sagittal sections. Anterior, right; dorsal, up. (e-h), Olfr558 expression in a knockout/reporter mouse in which the Olfr558 coding region is replaced with lacZ encoding β-galactosidase. (e), Olfr558 reporter expression in blood vessels of CB and SCG by X-gal staining. Heterozygous Olfr558^{+/lacZ} samples showed the same pattern of staining (data not shown). (f-h), CB sections immunostained for β-galactosidase (Olfr558 reporter; green), TH (red), with DAPI counterstain (blue) in (f), and for β-galactosidase (green) and CD31 (red) in (g) or smooth muscle actin (red) in (h). Scale bars, 100 μm ((a), (b)-right, (c)-right, (f-h)), 200 μm ((b)-left, (c)-left), 500 μm (e), and 2 mm (d).

FIG. 8. Tidal volume and minute ventilation of Olfr78^{-/-} mutants exposed to hypoxia and hypercapnia. Whole body plethysmography of unrestrained, unanesthetized Olfr78^{+/+} control and Olfr78^{-/-} mutant littermates (as in FIG. 2). (a, b), Tidal volume (TV) and minute ventilation (MV) of animals exposed to hypoxia. n=9 (+/+), 8 (−/−) animals. (c, d) TV and MV of animals exposed to hypercapnia. n=4 (+/+), 5 (−/−) animals. Data as mean±s.e.m. *P<0.05, ***P<0.001 by paired t test.

FIG. 9. Physiological responses of Olfr78^{-/-} mutants to hypoxia in vivo. (a-f), Arterial blood gas measurements of Olfr78^{-/-} control and Olfr78^{-/-} mutant animals exposed to hypoxia. PaO₂ (a), PaCO₂ (b), and pH (c) values of blood collected from the right carotid artery of anesthetized Olfr78^{+/+} control and Olfr78^{-/-} mutant animals exposed to normoxia (21% O₂) and hypoxia (10% O₂) for 3 min. Oxygen saturation (sO₂, d), [HCO₃ ⁻] (e), and base excess of extracellular fluid (BE_{ecf,} f) calculated from PaO₂ (a), PaCO₂ (b), and pH (c) values. n=4 (+/+, 21% O₂), 5 (−/−, 21% O₂), 4 (+/+, 10% O₂), 6 (−/−, 10% O₂) animals. (g), Body temperature of unanesthetized Olfr78^{+/+} control and Olfr78^{-/-} mutant littermates in room air (21% O₂) and exposed to hypoxia (10% O₂) for indicated times. n=4 (+/+), 6 (−/−) animals. (h-j), Metabolic values measured by indirect calorimetry of unanesthetized Olfr78^{+/+} control and Olfr78^{-/-} mutant littermates exposed to normoxia (21% O₂) and hypoxia (10% O₂) for 10 min. n=4 (+/+), 6 (−/−) animals. Data as mean±s.e.m. *P<0.05 by unpaired t test.

FIG. 10. Carotid body chemosensory responses assayed by carotid sinus nerve activity. (a, b), Raw discharge frequency (extracellular recording) of carotid sinus nerves from Olfr78^{+/+} control and Olfr78^{-/-} mutant animals at time 0 (a) and 9 minutes (b) after the change in gas bubbling the perfusion buffer from 95% O₂/5% CO₂ to 95% N₂/5% CO₂. c, d, Carotid sinus nerve activity of an Olfr78^{+/-} nerve 9 minutes after the change in gas to 95% N₂/5% CO₂ (c) and 2 minutes later after addition of 7.5 μM tetrodotoxin (TTX) while still bubbling 95% N₂/5% CO₂ (d). Scored action potentials are marked by filled circles. (e-h), Time course of carotid sinus nerve activity in the Olfr78 genotypes indicated scored using Spike2 software (e, g) or by hand (f, h) and showing mean±s.e.m (e, f) or individual (g, h) values. The residual responses of Olfr78^{-/-} nerves to hypoxia were more apparent when scored by hand. n=6 (3 +/+, 3 +/−), 5 (−/−) animals. *P<0.05, **P<0.01, ***P<0.001 by unpaired t test. Olfr78^{+/+} and Olfr78^{+/-} recordings were not significantly different from each other at any time point, except for time=11 min, by unpaired t test (P>0.05). (I, j), Time course of raw discharge of carotid sinus nerves from Olfr78^{+/+} control and Olfr78^{-/-} mutant animals in response to acetate (30 mM, 5 min), propionate (30 mM, 5 min), and lactate (30 mM, 5 and 10 min), and pH 7.0 (5 min) scored using Spike2 software (i) or by hand (j). Recovery times were 15 min between acetate, propionate, and lactate, and at least 30 min between lactate and pH 7.0. To minimize the contribution of endogenous hypoxic signals, the superperfusion buffer in the chamber was maintained at hyperoxic conditions (PO₂=625 mmHg). n=5 (+/+), 5 (−/−) animals. Data as mean±s.e.m. *P<0.05, **P<0.01 by unpaired t test.

FIG. 11. Lactate activates Olfr78 expressed in HEK293T cells and increases acutely in blood in hypoxia in vivo. (a, c), HEK293T cells transfected with empty vector pCl (a) or pCl-Rho-Olfr78 (b) and RTP1S (OR transport protein) and cytoplasmic GFP (co-transfection marker) plasmids. Transfected cells were stained before fixation to detect Rho-tagged Olfr78 (anti-Rho; red) on the cell surface. GFP (transfection marker, green); DAPI (nuclei, blue). Bar, 100 μm. (c), Quantitation of cells expressing GFP and Rho as percentage of DAPI-positive cells in fields shown in (a) and (b). n=164 (pCl), 108 (pCl-Rho-Olfr78) cells. Data as percent±standard error of percentage. (d), Dose-response curves for propionate, acetate, and chloride compared to lactate in activation of Olfr78 in transfected HEK293T cells as in FIG. 4a. n=8 (propionate), 12 (acetate), 4 (chloride), and 12 (lactate) wells. Data as mean±s.e.m. By analysis of variance (ANOVA), all chemicals except chloride (P=0.309) showed significant difference (P<0.001). (e), Dose-response curves as in (c) except cells were transfected with empty vector (pCl). ANOVA showed no significant difference (P>0.05) for any chemical. (f), EC₅₀, 95% confidence interval of EC₅₀, and relative maximal activation values from fitted curves in (c). ND, not determined due to lack of curve fitting to data. (g), Structures of the short-chain fatty acids. (h), Lactate concentrations in blood collected from tail artery of restrained, unanesthetized Offr78^{+/+} control and Olfr78^{-/-} mutant littermates exposed to hypoxia (10% O₂) for 4-5 min. Values for animals in normoxia (21% O₂) are likely to be an overestimate of baseline concentrations due to greater restraint required to immobilize animals in normoxia. n=5 (+/+), 6 (−/−) animals. Data as mean±s.e.m. *P<0.05 by paired t test.

FIG. 12. Calcium imaging of responses of carotid body glomus cells to chemosensory stimuli. (a), Carotid body (CB) of a Th-Cre; ROSA-tdTomato adult immunostained for the Cre-dependent reporter tdTomato (red) and TH (green) to show glomus cells and counterstained with DAPI (nuclei, blue). tdTomato labeled glomus cells. (b-e), Tissue preparations for calcium imaging of CBs from TH-Cre; ROSA-GCaMP3 animals that express the calcium indicator GCaMP3 selectively in glomus cells. (b), DIC image of whole mount carotid bifurcation with GCaMP3 fluorescence pseudocolored green. (c), High magnification, two-photon image of boxed region in (b). (d), DIC image of CB tissue slice with GCaMP3 fluorescence pseudocolored green. (e), Two-photon image of CB slice in (d). Inset shows glomus cell marked by asterisk at higher magnification. GCaMP3 fluorescence was seen in cytoplasm and excluded from nucleus of glomus cells. Bars, 100 μm (a), 200 μm (b), 50 μm (c-e). (f-i) Time course of calcium responses of individual glomus cells to hypoxia, lactate, and cyanide. Whole mount CBs were exposed sequentially to hypoxia (40-50 mmHg), lactate (30 mM), and cyanide (2 mM). Interval between data points is ˜2 minutes, the time required to acquire a stack of images through the CB, excluding the 2 minute ramp times between stimuli. All glomus cells analyzed (n=42 cells) responded strongly to cyanide. Fluorescence traces shown are the 29 individual glomus cells that responded to both hypoxia and lactate, arranged in order of decreasing initial fluorescence intensity. The other 13 glomus cells responded to either hypoxia (9 cells) or lactate (4 cells). Multiple data points for buffer or stimuli were averaged to generate the data presented in FIG. 4c. Background colors match bar colors in FIG. 4d.

### DETAILED DESCRIPTION OF THE EMBODIMENTS

The following detailed description refers to the accompanying drawings which show, by way of illustration, specific aspects and embodiments in which the present invention may be practiced. The various embodiments are not necessarily mutually exclusive, as aspects of one embodiment can be combined with aspects of another embodiment. These embodiments are described in sufficient detail to enable those skilled in the art to practice the invention. Other embodiments may be utilized and structural, logical, and electrical changes may be made without departing from the scope of the present invention.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Olfr78/OR51E2 and Olfr558 are two members of a family of olfactory receptors that contains three olfactory receptors, which are a subfamily of G protein-coupled receptors. The human counterpart gene, OR51E2, is also referred to as PSGR. The protein is 320 amino acids, contains a predicted N-terminal N-glycosylation site, conserved cysteine residues in the first 2 extracellular loops, and conserved sequence motifs in addition to the 7 transmembrane domain GPCR signature motif, and shares approximately 50% amino acid identity to the G protein-coupled odorant receptor family. It has been reported to be expressed in prostate cancer cells. The OR51E2 gene contains 2 exons separated by a 14.9-kb intron. The genetic sequence of the human counterpart may be accessed at Genbank, NM_030774.

“OLFR78/OR51E2 agonists” include molecules, i.e. ligands, that bind to and activate the Olfr78/OR51E2, which ligands may activate the receptor (agonist) or inhibit the receptor (antagonist). In some embodiments, the ligands selected for use in the methods of the invention have an EC50 of not more than about 10 mM, not more than about 5 mM, not more than about 2.5 mM, not more than about 1 mM, not more than about 500 mM, not more than about 250 mM, not more than about 100 mM, not more than about 50 mM, not more than about 24 mM, not more than about 1 mM.

Molecules useful as OLFR78/OR51E2 agonists include derivatives, variants, and biologically active fragments of known activators, including for example lactate, proprionate, β-ionone, steroids, including 6-dehydrotestosterone, ADT, 1,4,6-androstatriene-17β-ol -3-one, short chain fatty acids, and derivatives and analogs thereof.

Suitable OLFR78/OR51E2 agonists or antagonists may be identified by compound screening by detecting the ability of an agent to activate OLFR78/OR51E2. In vitro assays may be conducted as a first screen for efficacy of a candidate agent, and usually an in vivo assay will be performed to confirm the biological assay. Desirable agents are temporary in nature, e.g. due to biological degradation.

In vitro assays for OLFR78/OR51E2 biological activity include, e.g. membrane depolarization, release of neurotransmitters, altered ventilation, Ca++ uptake, and the like. A candidate agent useful as a OLFR78/OR51E2 agonist results in the down upregulation of activity, e.g. Ca++ release, etc. by at least about 10%, at least about 20%, at least about 50%, at least about 70%, at least about 80%, or up to about 90% compared to level observed in absence of candidate agent.

Polynucleotide encoding soluble OLFR78/OR51E2 or soluble OLFR78/OR51E2-Fc can be introduced into a suitable expression vector. The expression vector is introduced into a suitable cell. Expression vectors generally have convenient restriction sites located near the promoter sequence to provide for the insertion of polynucleotide sequences. Transcription cassettes may be prepared comprising a transcription initiation region, OLFR78/OR51E2 gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks. The various manipulations may be carried out in vitro or may be performed in an appropriate host, e.g. E. coli, mammalian cells, etc.

OLFR78/OR51E2 can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, protein G affinity chromatography, for example, hydroxylapatite chromatography and lectin chromatography. Most preferably, high performance liquid chromatography (“HPLC”) is employed for purification. Alternatively, assays are performed on cells engineered to express OLFR78/OR51E2.

A plurality of assays may be run in parallel with different concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in binding.

Compounds of interest for screening include biologically active agents of numerous chemical classes, primarily organic molecules, although including in some instances inorganic molecules, organometallic molecules, immunoglobulins, chimeric OLFR78/OR51E2 proteins, OLFR78/OR51E2 related proteins, genetic sequences, etc. Also of interest are small organic molecules, which comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Compounds are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

A chemical screen for agonists of OR51E2, OR51E1, and Olfr558 using 93 named compounds found that OR51E2 was specifically activated by propionic acid, OR51E1 by nonanoic acid, and butyl butylryllactate, and Olfr558 by nonanoic acid. Another study found that 4-methyl-valeric acid and 3-methyl-valeric acid and 3 other similar small fatty acids were agonists for OR51E1 while longer chain fatty acids and alcohols were not good agonists. A more recent study shows that OR51E1 can be activated by propionic acid, isovaleric acid, and nonanoic acid. In a study looking at Olfr78 and OR51E2, acetate (2 carbons) and propionate (3 carbons) were identified as agonists for both receptors and not longer and shorter straight chain fatty acids like formate (1 carbon) and butyrate (4 carbons). Olfr78 is activated by lactate (2-hydroxypropanoic acid), a physiologically relevant metabolite produced in low oxygen.

Olfr78 and related receptors are activated by short chain fatty acids. Olfr78/OR51E2 and Olfr558/OR51E1 have overlapping activation profiles (propionate). Olfr78/OR51E2 is perhaps activated more by shorter chain fatty acids of 2-3 straight chain carbons (acetate and propionate) and Olfr558/OR51E1 by longer chain fatty acids of up to 9 straight chain carbons (nonanoic acid). Because the conjugate base alone can stimulate receptor activity, the evidence suggests that it is the conjugate base that is relevant and not the undissociated acid or H⁺ ion.

OLFR78/OR51E2 inhibitors. Agents of interest as OLFR78/OR51E2 inhibitors include specific binding members that inhibit the signaling activity of OLFR78/OR51E2. The term “specific binding member” or “binding member” as used herein refers to a member of a specific binding pair, i.e. two molecules, usually two different molecules, where one of the molecules (i.e., first specific binding member) through chemical or physical means specifically binds to the other molecule (i.e., second specific binding member). OLFR78/OR51E2 inhibitors useful in the methods of the invention include analogs, derivatives and fragments of the original specific binding member.

In some embodiments, the specific binding member is an antibody. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. Antibodies utilized in the present invention may be polyclonal antibodies, although monoclonal antibodies are preferred because they may be reproduced by cell culture or recombinantly, and can be modified to reduce their antigenicity.

Polyclonal antibodies can be raised by a standard protocol by injecting a production animal with an antigenic composition. See, e.g., Harlow and Lane, Antibodies: A Laboratory Manual, Cold Spring Harbor Laboratory, 1988. Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line. In addition, the antibodies or antigen binding fragments may be produced by genetic engineering. Humanized, chimeric, or xenogeneic human antibodies, which produce less of an immune response when administered to humans, are preferred for use in the present invention.

In addition to entire immunoglobulins (or their recombinant counterparts), immunoglobulin fragments comprising the epitope binding site (e.g., Fab′, F(ab′)₂, or other fragments) are useful as antibody moieties in the present invention. Such antibody fragments may be generated from whole immunoglobulins by ricin, pepsin, papain, or other protease cleavage. “Fragment,” or minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif).

The efficacy of a OLFR78/OR51E2 inhibitor is assessed by assaying OLFR78/OR51E2 activity. The above-mentioned assays or modified versions thereof are used. An inhibitor OLFR78/OR51E2 will counteract activation by at least about 10%, or up to 20%, or 50%, or 70% or 80% or up to about 90% compared to the level of activity in absence of the candidate agent.

In one embodiment of the invention, the agent, or a pharmaceutical composition comprising the agent, is provided in an amount effective to detectably modulate ventilation. The effective amount is determined via empirical testing routine in the art.

An “individual” is a vertebrate, preferably a mammal, more preferably a human. Mammals include, but are not limited to, rodents, primates, farm animals, sport animals, and pets. The terms “recipient”, “individual”, “subject”, “host”, and “patient”, used interchangeably herein and refer to any mammalian subject for whom diagnosis, treatment, or therapy is desired, particularly humans.

An “effective amount” is an amount sufficient to effect beneficial or desired clinical results. An effective amount can be administered in one or more administrations. For purposes of this invention, an effective amount of an inhibitor or agonist of OR51E2 is an amount that is sufficient to palliate, ameliorate, stabilize, reverse, slow or delay the progression of the disease state by modulating breathing of the subject.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, preventing spread (i.e., metastasis) of disease, delay or slowing of disease progression, amelioration or palliation of the disease state, and remission (whether partial or total), whether detectable or undetectable. “Treatment” can also mean prolonging survival as compared to expected survival if not receiving treatment. “Palliating” a disease means that the extent and/or undesirable clinical manifestations of a disease state are lessened and/or time course of the progression is slowed or lengthened, as compared to not administering the methods of the present invention.

“Therapeutic target” refers to a gene or gene product that, upon modulation of its activity (e.g., by modulation of expression, biological activity, and the like), can provide for modulation of the cancerous phenotype. As used throughout, “modulation” is meant to refer to an increase or a decrease in the indicated phenomenon (e.g., modulation of a biological activity refers to an increase in a biological activity or a decrease in a biological activity).

Hypoxic hypoxia, stagnant, or ischemic hypoxia and histotoxic hypoxia are powerful stimuli of the carotid body. Transient episodes of hypoxia are associated with many pathophysiological situations, including sleep-disordered breathing with recurrent apneas (obstructive or central apneas). Recurrent apneas are prevalent in premature infants, 5% of middle-aged men and 2% of women after menopause and the incidence of recurrent apneas is increasing in adults. In severely affected patients, the frequency of apneas may exceed as many as 60 episodes/h and arterial blood O₂ saturation can be reduced to as low as 50%. As a consequence of periodic cessations of breathing, patients with recurrent apneas experience not only chronic intermittent hypoxia (IH) but also chronic intermittent hypercapnia (i.e., elevations in arterial CO₂). Although both continuous and IH results in decreases in arterial O₂, physiological responses to both forms of hypoxia differ quite considerably. Whilst continuous hypoxia leads to adaptations of the physiological systems, chronic IH associated with recurrent apneas result in morbidities including development of hypertension, elevated sympathetic nerve activity, myocardial infarctions, and stroke. Evidence suggests that chronic IH sensitizes carotid body response to acute hypoxia. Chronic IH also induces plasticity manifested as sensory long-term facilitation of the carotid body. This long-lasting increase in baseline sensory activity has been termed sensory LTF because it resembled the time course of LTF of breathing elicited by repetitive hypoxia, although control carotid bodies do not exhibit (or have a weak) sensory LTF in response to repetitive hypoxia.

Depending on gestational age, nearly 90% of infants born preterm experience IH as a consequence of recurrent apneas. Carotid bodies are immature at birth and respond poorly to hypoxia compared with adults. However, preterm infants exhibiting greater number of apneas display augmented peripheral chemoreceptor reflex as evidenced by more pronounced ventilatory depression in response to brief hyperoxia (Dejour's test) than those having lesser incidence of apneas. Observations suggest that exposure to IH, as short as few hours, induces hypoxic sensitivity of the carotid bodies in neonates, which otherwise are insensitive to hypoxia. IH sensitizes carotid body response to hypoxia in neonates as it does in adults. In contrast to adults, sensitization of the carotid body response to hypoxia was not reversed in neonates after reexposure to normoxia. Rather they persisted in juvenile life. Carotid bodies from neonates appear more sensitive to IH than in adults. Treatment of pre-term IH may include administration of an effective dose of an OLFR78/OR51E2 agonist.

Congestive heart failure (CHF) is a major health care problem affecting one in 10 men and women after age 60 in the USA alone. Sympathohumoral activation is a hallmark of CHF, which contributes to the progression and ultimate mortality of the disease. The role of chemoreflexes arising from the carotid body in sympathetic activation in CHF has received considerable attention in recent years.

CHF patients, especially those in more severe stages of heart failure display exaggerated peripheral chemoreflex function as evidenced by augmented ventilatory response to hypoxia. Animal models have provided clear evidence for the contribution of the carotid chemoreflex to sympathetic activation in CHF. Treatment methods for CHF may include administration of an effective dose of a OLFR78/OR51E2 inhibitor.

Patients with essential hypertension exhibit enhanced sympathetic nerve and ventilatory response to hypoxia, and these responses were attributed to an exaggerated carotid body response to low O₂. Enlargement of the carotid body was reported in humans with essential but not with renal hypertension. Treatment methods for hypertension may include administration of an effective dose of a OLFR78/OR51E2 inhibitor.

Sleep apnea is defined as an intermittent cessation of airflow at the nose and mouth during sleep. By convention, apneas of at least 10 seconds in duration have been considered important, but in most individuals the apneas are 20-30 seconds in duration and may be as long as 2-3 minutes. While there is some uncertainty as to the minimum number of apneas that should be considered clinically important, by the time most individuals come to attention of the medical community they have at least 10 to 15 events per hour of sleep.

Sleep apneas have been classified into three types: central, obstructive, and mixed. In central sleep apnea the neural drive to all respiratory muscles is transiently abolished. In obstructive sleep apneas, airflow ceases despite continuing respiratory drive because of occlusion of the oropharyngeal airway. Mixed apneas, which consist of a central apnea followed by an obstructive component, are a variant of obstructive sleep apnea. The most common type of apnea is obstructive sleep apnea.

Obstructive sleep apnea syndrome (OSAS) has been identified in as many as 24% of working adult men and 9% of similar women, with peak prevalence in the sixth decade. Habitual heavy snoring, which is an almost invariant feature of OSAS, has been described in up to 24% of middle aged men, and 14% of similarly aged women, with even greater prevalence in older subjects.

Obstructive sleep apnea syndrome's definitive event is the occlusion of the upper airway, frequently at the level of the oropharynx. The resultant apnea generally leads to a progressive-type asphyxia until the individual is briefly aroused from the sleeping state, thereby restoring airway patency and thus restoring airflow.

The recurrent episodes of nocturnal asphyxia and of arousal from sleep that characterize OSAS lead to a series of secondary physiologic events, which in turn give rise to the clinical complications of the syndrome. The most common manifestations are neuropsychiatric and behavioral disturbances that are thought to arise from the fragmentation of sleep and loss of slow-wave sleep induced by the recurrent arousal responses. Nocturnal cerebral hypoxia also may play an important role. The most pervasive manifestation is excessive daytime sleepiness. OSAS is now recognized as a leading cause of daytime sleepiness and has been implicated as an important risk factor for such problems as motor vehicle accidents. Other related symptoms include intellectual impairment, memory loss, personality disturbances, and impotence.

The other major manifestations are cardiorespiratory in nature and are thought to arise from the recurrent episodes of nocturnal asphyxia. Most individuals demonstrate a cyclical slowing of the heart during the apneas to 30 to 50 beats per minute, followed by tachycardia of 90 to 120 beats per minute during the ventilatory phase. A small number of individuals develop severe bradycardia with asystoles of 8 to 12 seconds in duration or dangerous tachyarrhythmias, including unsustained ventricular tachycardia. OSAS also aggravates left ventricular failure in patients with underlying heart disease. This complication is most likely due to the combined effects of increased left ventricular afterload during each obstructive event, secondary to increased negative intrathoracic pressure, recurrent nocturnal hypoxemia, and chronically elevated sympathoadrenal activity.

Central sleep apnea is less prevalent as a syndrome than OSAS, but can be identified in a wide spectrum of patients with medical, neurological, and/or neuromuscular disorders associated with diurnal alveolar hypoventilation or periodic breathing. The definitive event in central sleep apnea is transient abolition of central drive to the ventilatory muscles. The resulting apnea leads to a primary sequence of events similar to those of OSAS. Several underlying mechanisms can result in cessation of respiratory drive during sleep. First are defects in the metabolic respiratory control system and respiratory neuromuscular apparatus. Other central sleep apnea disorders arise from transient instabilities in an otherwise intact respiratory control system.

In individuals with clinically significant central sleep apnea, the primary sequence of events that characterize the disorder leads to prominent physiological and clinical consequences. In those individuals with central sleep apnea alveolar hypoventilation syndrome, daytime hypercapnia and hypoxemia are usually evident and the clinical picture is dominated by a history of recurrent respiratory failure, polycythemia, pulmonary hypertension, and right-sided heart failure. Complaints of sleeping poorly, morning headache, and daytime fatigue and sleepiness are also prominent. In contrast, in individuals whose central sleep apnea results from an instability in respiratory drive, the clinical picture is dominated by features related to sleep disturbance, including recurrent nocturnal awakenings, morning fatigue, and daytime sleepiness.

Treatment of sleep apnea may include administration of an effective dose of an OLFR78/OR51E2 agonist

### Pharmaceutical Formulations

A “delayed release dosage form” is one that releases a drug (or drugs) at a time other than promptly after administration.

An “extended release dosage form” is one that allows at least a twofold reduction in dosing frequency as compared to the drug presented as a conventional dosage form (e.g. as a solution or prompt drug-releasing, conventional solid dosage form).

A “pulsatile release dosage form” is one that mimics a multiple dosing profile without repeated dosing and allows at least a twofold reduction in dosing frequency as compared to that drug presented as a conventional dosage form (e.g. as a solution or prompt drug-releasing, conventional solid dosage form). A pulsatile release profile is characterized by a time period of no release (lag time) followed by rapid drug release.

A “modified release dosage form” is one for which the drug release characteristics of time, course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms. The pharmaceutical combination of the invention may have any or all of its constituents in a modified release dosage form. A “modified release pharmaceutical composition” has at least one of its components in modified release dosage form.

As used herein “active compounds” in addition to their free base and quaternized forms also encompasses pharmaceutically acceptable, pharmacologically active derivatives of active compounds including individual enantiomers and their pharmaceutically acceptable salts, mixtures of enantiomers and their pharmaceutically acceptable salts, and active metabolites of active compounds and their pharmaceutically acceptable salts, unless otherwise noted. It is understood that in some cases dosages of enantiomers, derivatives, and metabolites may need to be adjusted based on relative activity of the racemic mixture of active compound.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxyrnaleic, phenylacetic, glutamic, benzoic, salicylic, sulfanilic, 2-acetoxybenzoic, fumaric, toluenesulfonic, methanesulfonic, ethane disulfonic, oxalic, and isethionic.

The pharmaceutically acceptable salts of the compounds can be synthesized from the parent compound, which contains a basic or acidic moiety, by conventional chemical methods.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, the term “stereoisomers” refers to compounds made up of the same atoms bonded by the same bonds but having different spatial structures which are not interchangeable. The three-dimensional structures are called configurations. As used herein, the term “enantiomers” refers to two stereoisomers whose molecules are nonsuperimposable mirror images of one another. As used herein, the term “optical isomer” is equivalent to the term “enantiomer”. The terms “racemate”, “racemic mixture” or “racemic modification” refer to a mixture of equal parts of enantiomers. The term “chiral center” refers to a carbon atom to which four different groups are attached. The term “enantiomeric enrichment” as used herein refers to the increase in the amount of one enantiomer as compared to the other. Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art using standard techniques well known in the art, such as those described by J. Jacques, et al., “Enantiomers, Racemates, and Resolutions”, John Wiley and Sons, Inc., 1981. Examples of resolutions include recrystallization of diastereomeric salts/derivatives or preparative chiral chromatography.

The pharmaceutical compositions of the invention can be administered adjunctively with other active compounds such as analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti asthma drugs, cardiovascular drugs, corticosteroids, dopaminergics, electrolytes, gastrointestinal drugs, muscle relaxants, nutritional agents, vitamins, parasympathomimetics, stimulants, anorectics and anti-narcoleptics.

By adjunctive administration is meant simultaneous administration of the compounds, in the same dosage form, simultaneous administration in separate dosage forms, and separate administration of the compounds.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes but is not limited to diluents, binders, lubricants, disintegrators, fillers, and coating compositions.

“Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, .sup.6th Edition, Ansel et. al., (Media, Pa.: Williams and Wilkins, 1995) which provides information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit™. (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer.™. 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

The immediate release dosage unit of the dosage form—i.e., a tablet, a plurality of drug-containing beads, granules or particles, or an outer layer of a coated core dosage form—contains a therapeutically effective quantity of the active agent with conventional pharmaceutical excipients. The immediate release dosage unit may or may not be coated, and may or may not be admixed with the delayed release dosage unit or units (as in an encapsulated mixture of immediate release drug-containing granules, particles or beads and delayed release drug-containing granules or beads).

Each dosage form contains a therapeutically effective amount of active agent. The effective dose may be from about 0.1 μg to about 1 g/kg weight of the subject. For dosage forms that mimic the twice daily dosing profile, approximately 30 wt. % to 80 wt. %, preferably 40 wt. % to 70 wt. %, of the total amount of active agent in the dosage form is released in the initial pulse, and, correspondingly approximately 70 wt. % to 20 wt. %, preferably 60 wt. % to 30 wt. %, of the total amount of active agent in the dosage form is released in the second pulse. For dosage forms mimicking the twice daily dosing profile, the second pulse is preferably released approximately 3 hours to less than 14 hours, and most preferably approximately 5 hours to 12 hours, following administration.

A kit is provided wherein the pharmaceutical composition of the invention is packaged accompanied by instructions. The packaging material may be a box, bottle, blister package, tray, or card. The kit will include a package insert instructing the patient to take a specific dose at a specific time, for example, a first dose on day one, a second dose on day two, a third dose on day three, and so on, until a maintenance dose is reached.

As will be appreciated by those skilled in the art and as described in the pertinent texts and literature, a number of methods are available for preparing drug-containing tablets, beads, granules or particles that provide a variety of drug release profiles. Such methods include, but are not limited to, the following: coating a drug or drug-containing composition with an appropriate coating material.

The methods illustrated in this disclosure are not intended to be exclusive of other methods within the scope of the present subject matter. Those of ordinary skill in the art will understand, upon reading and comprehending this disclosure, other methods within the scope of the present subject matter. The above-identified embodiments, and portions of the illustrated embodiments, are not necessarily mutually exclusive. These embodiments, or portions thereof, can be combined. In various embodiments, the methods provided above are implemented as a computer data signal embodied in a carrier wave or propagated signal, that represents a sequence of instructions which, when executed by a processor cause the processor to perform the respective method. In various embodiments, methods provided above are implemented as a set of instructions contained on a computer-accessible medium capable of directing a processor to perform the respective method. In various embodiments, the medium is a magnetic medium, an electronic medium, or an optical medium.

Although specific embodiments have been illustrated and described herein, it will be appreciated by those of ordinary skill in the art that any arrangement which is calculated to achieve the same purpose may be substituted for the specific embodiment shown. This application is intended to cover adaptations or variations of the present subject matter. It is to be understood that the above description is intended to be illustrative, and not restrictive. Combinations of the above embodiments as well as combinations of portions of the above embodiments in other embodiments will be apparent to those of skill in the art upon reviewing the above description. The scope of the present subject matter should be determined with reference to the appended claims, along with the full scope of equivalents to which such claims are entitled.

### EXAMPLE 1

### Oxygen Control of Breathing by an Olfactory Receptor Activated by Lactate

Animals have evolved homeostatic responses to changes in oxygen availability that act on different time scales. Although the hypoxia-inducible factor (HIF) transcriptional pathway that controls long term responses to low oxygen (hypoxia) has been established, the pathway that mediates acute responses to hypoxia in mammals is not well understood. Here we show that the olfactory receptor Olfr78 is highly and selectively expressed in oxygen-sensitive glomus cells of the carotid body, a chemosensory organ at the carotid artery bifurcation that monitors blood oxygen and stimulates breathing within seconds when oxygen declines. Olfr78 mutants fail to increase ventilation in hypoxia but respond normally to hypercapnia. Glomus cells are present in normal numbers and appear structurally intact, but hypoxia-induced carotid body activity is diminished. Lactate, a metabolite that rapidly accumulates in hypoxia and induces hyperventilation, activates Olfr78 in heterologous expression experiments, induces calcium transients in glomus cells, and stimulates carotid sinus nerve activity through Olfr78. In addition to its role in olfaction, Olfr78 acts as a hypoxia sensor in the breathing circuit by sensing lactate produced when oxygen levels decline.

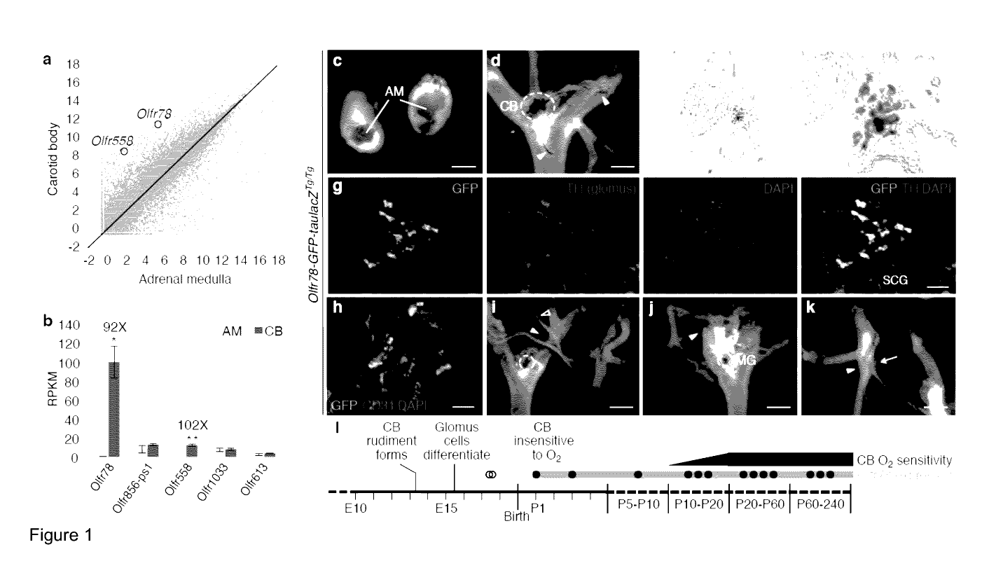

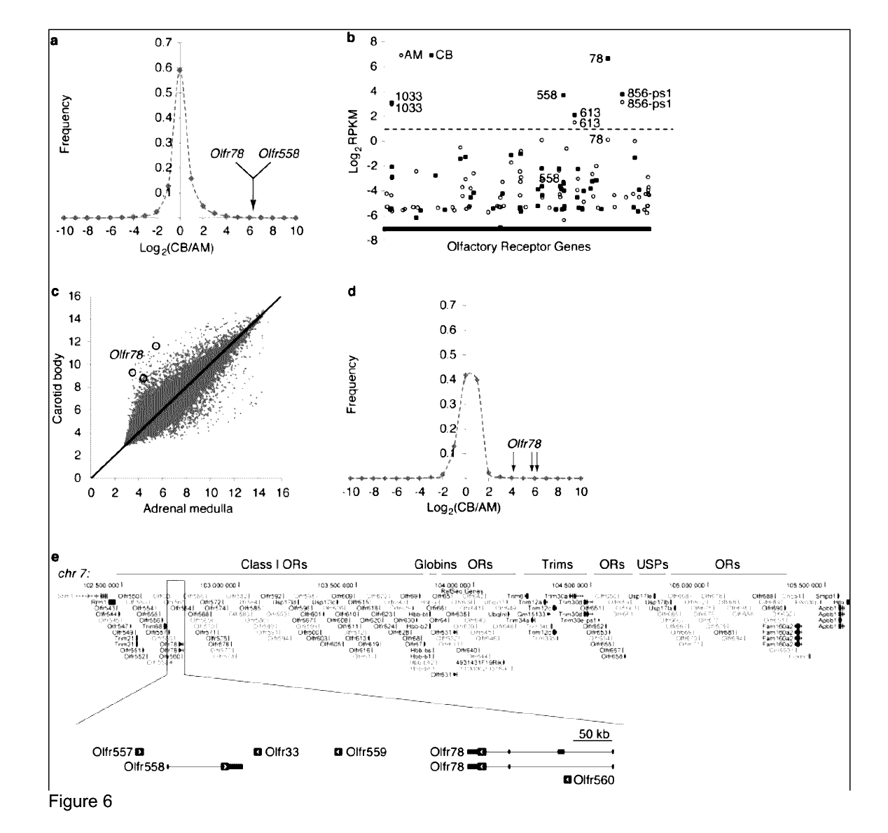

To identify new candidate molecules involved in carotid body oxygen sensing, we used RNA sequencing and whole genome microarrays to compare gene expression of the carotid body from wild-type adult C57BL/6J mice to that of the adrenal medulla, which shares developmental and functional similarities with the carotid body but does not respond acutely to hypoxia. We reasoned that an oxygen sensor would be expressed at high levels in carotid body relative to adrenal medulla, and focused on signaling molecules that can act on the acute time scale of carotid body sensing. Transcripts for the olfactory receptor Olfr78 were highly expressed in carotid body (top 4% of all genes by RNA sequencing) and highly enriched relative to adrenal medulla by both RNA sequencing (92-fold) and microarrays (3 probe sets, 17 to 80-fold) (FIGS. 1a, b, FIGS. 6a-d, and Table 1).

Olfactory receptors (ORs) comprise a subfamily of G protein-coupled receptors that is the largest gene family in vertebrates, encoded by ˜1,200 genes in mouse. ORs are expressed in olfactory sensory neurons and detect volatile odorants in smell. However, some ORs are expressed in other tissues. The RNA sequencing results showed that three other OR genes (Olfr1033, Olfr613, and Olfr856ps1) were expressed (RPKM>2) in the carotid body, but at similar levels in adrenal medulla and thus not pursued further (FIG. 1b and FIG. 6b). Olfr558 was highly and selectively expressed in the carotid body, but at lower levels than Olfr78 (FIGS. 1a, b). Olfr78 and Olfr558 encode closely related proteins of the same OR subfamily and lie in close proximity in the genome (FIG. 6e). Because of the high and selective expression of Olfr78 and Olfr558 in the carotid body, we investigated their expression and potential function.

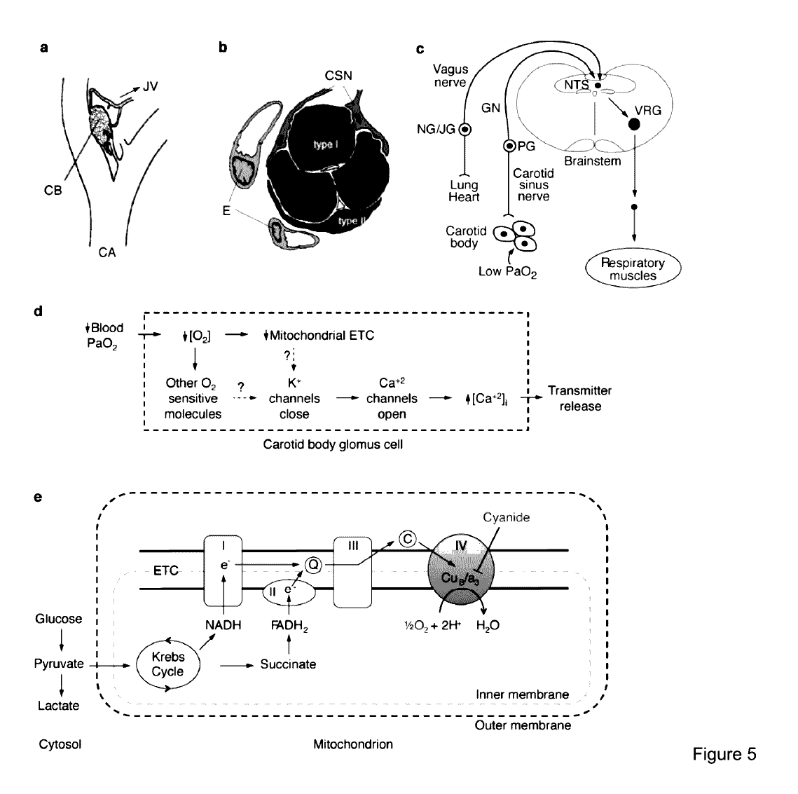

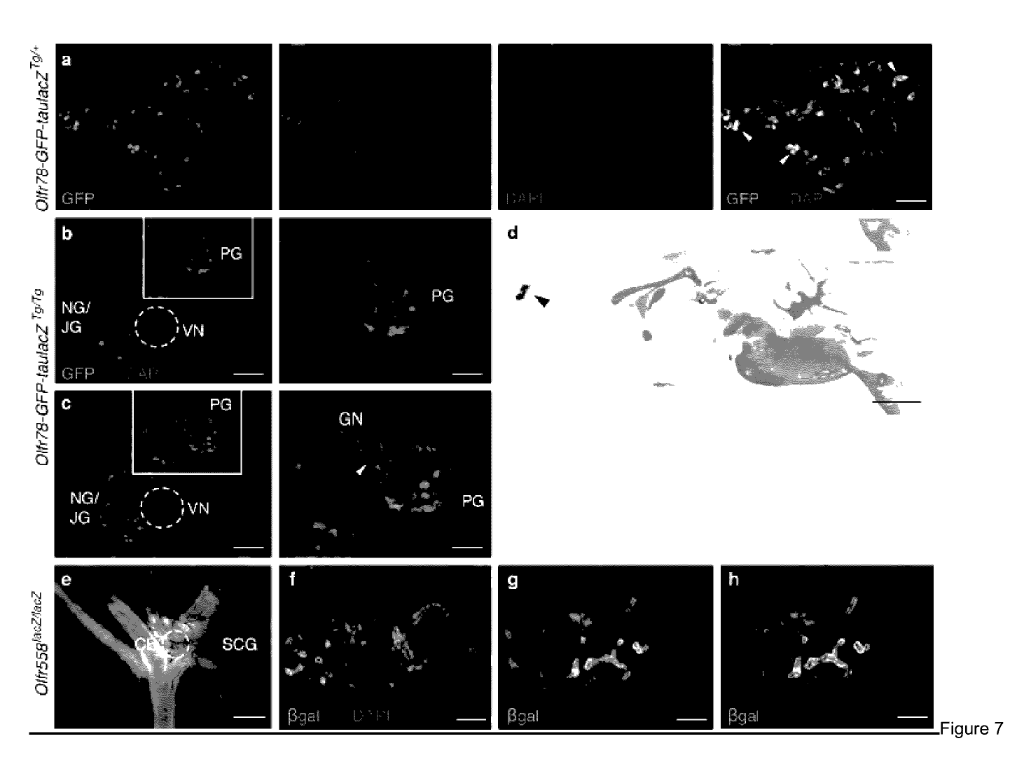

The carotid body is composed of Type I glomus cells that sense changes in oxygen, Type II sustentacular cells that resemble neuroglia, nerve fibers, and endothelial and smooth muscle cells that comprise fine tortuous vessels off the carotid artery (FIG. 5b). To determine which cells express Olfr78, we used an Olfr78 reporter strain carrying GFP and taulacZ genes in the 3′-untranslated region (3′-UTR) of the Olfr78 locus. X-gal staining for lacZ activity in adults confirmed strong and selective Olfr78 expression in carotid body and no detectable adrenal gland expression (FIGS. 1c-f). The cluster-like pattern of X-gal staining in carotid body suggested Olfr78 is expressed in glomus cells (FIGS. 1d-f). This was verified by antibody staining showing co-localization of Olfr78 reporter GFP with tyrosine hydroxylase (TH), a glomus cell marker (FIGS. 1g, h); 98% of all GFP and TH-positive cells expressed both markers (n=3 sections from 3 animals, 222 cells). Unlike the monoallelic expression of olfactory receptors observed in olfactory neurons, we found that in animals carrying only one allele of the Olfr78 reporter gene, 98% of all GFP and TH-positive cells still expressed both markers (n=3 sections from 3 animals, 271 cells, P=0.461 Tg/+vs. Tg/Tg by unpaired t test) (FIG. 7a). Using an Olfr558 lacZ knock-in reporter allele, we detected reporter activity in some vascular smooth muscle cells of carotid body blood vessels, but not in glomus cells (FIGS. 7e-h). Thus we focused on Olfr78.

Although Olfr78 and its human ortholog OR51E2 are expressed in other tissues outside the olfactory system, no expression of Olfr78 reporter was detected in other parts of the oxygen-sensing circuit for breathing besides carotid body (FIG. 5c): carotid sinus and glossopharyngeal nerves (FIGS. 1i-k), petrosal, nodose/jugular, and superior cervical ganglia (FIGS. 1i-k and FIGS. 7b, c), and brainstem (FIG. 7d). In some carotid bifurcations, there were patches of Olfr78-expressing cells on arteries that were innervated by branches of the glossopharyngeal nerve distinct from the carotid sinus nerve (FIG. 1j); these may be ectopic “miniglomera” with chemosensory functions similar to carotid body. We conclude that Olfr78 is specifically expressed in acute oxygen-sensing cells of the carotid body and not in afferent pathways or the respiratory centers themselves.

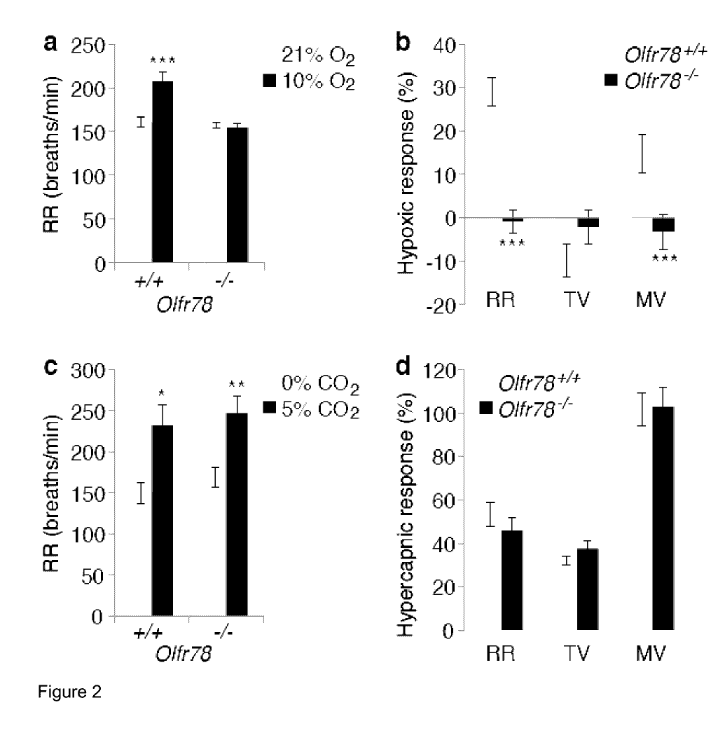

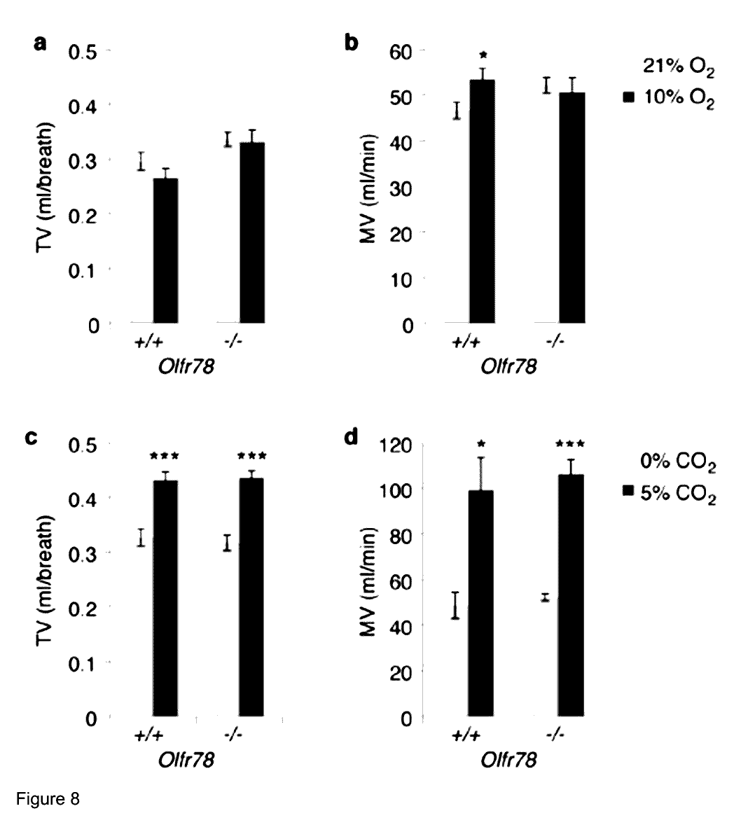

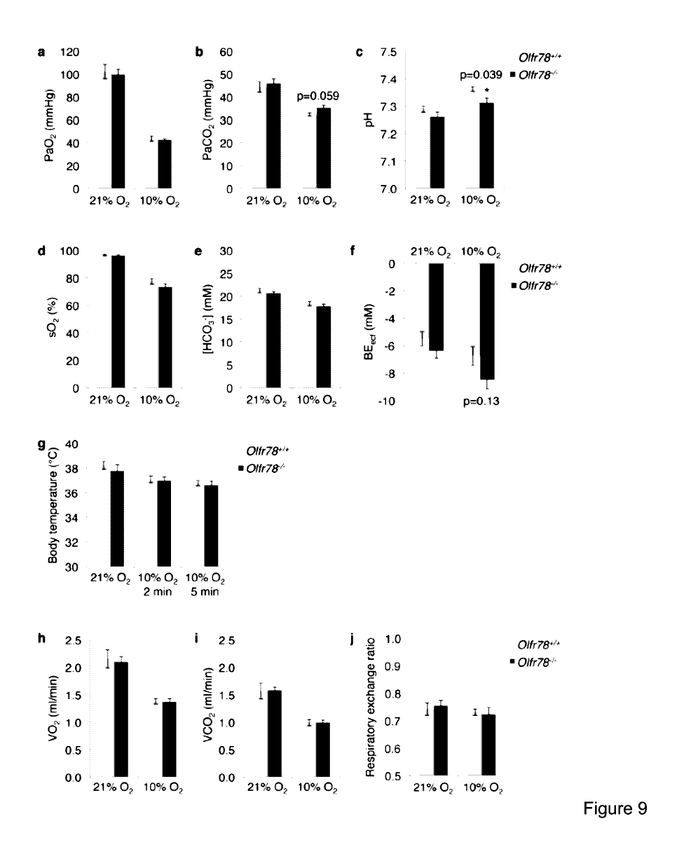

Because ORs mediate acute sensory signaling in olfaction, we tested whether Olfr78 was involved in acute oxygen sensing in the carotid body by examining breathing of Olfr78 knockout mice. Homozygous Olfr78^{-/-} mutants were viable and present in Mendelian ratios at birth (postnatal day 1 (P1); 15 +/+:35 +/−:19 −/−, X²=0.4783, P>0.7) and weaning (P21; 50 +/+:115 +/−:64 −/−, _{X}2=1.7162, P>0.3), and they appeared to breathe and behave normally under ambient conditions. However, when challenged by hypoxia (10% O₂), Olfr78^{+/+} control animals increased respiratory rate and minute ventilation, whereas Olfr78 ^{-/-} mutants did not exhibit significant ventilatory changes (FIGS. 2a, b and FIGS. 8a, b). Most strikingly, the respiratory rate of Olfr78 ^{-/-} mutants did not change in hypoxia, while increasing ˜30% in controls (FIGS. 2a, b). In hypoxia, arterial blood from Olfr78 −/− mutants had higher PaCO₂ and lower pH than wild-type animals (FIGS. 9a-f), consistent with their inability to increase ventilation. By contrast, ventilatory responses to hypercapnia (5% CO₂) remained intact in Olfr78^{-/-} mutants (FIGS. 2c, d and FIGS. 8c, d), as did two other rapid behavioral responses to hypoxia: reduced locomotion and more regular breathing. We also did not detect differences between controls and Olfr78^{-/-} mutants in body temperature or metabolism in response to hypoxia (FIGS. 9g-j), parameters that can affect hypoxic ventilation in small mammals. Thus, Olfr78^{-/-} mutants have a specific defect in hypoxic regulation of respiratory rate, a physiological function controlled by the carotid body.

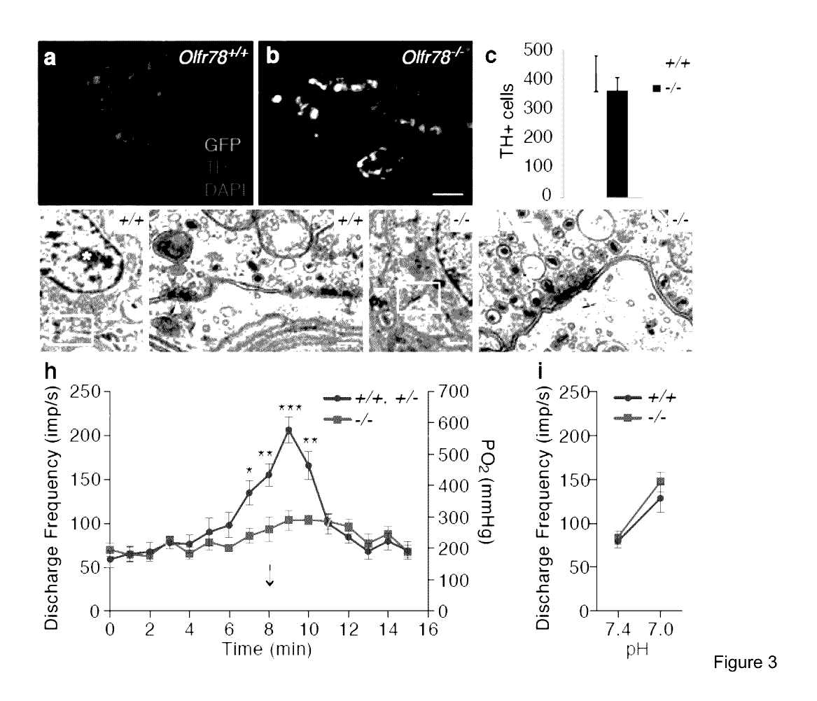

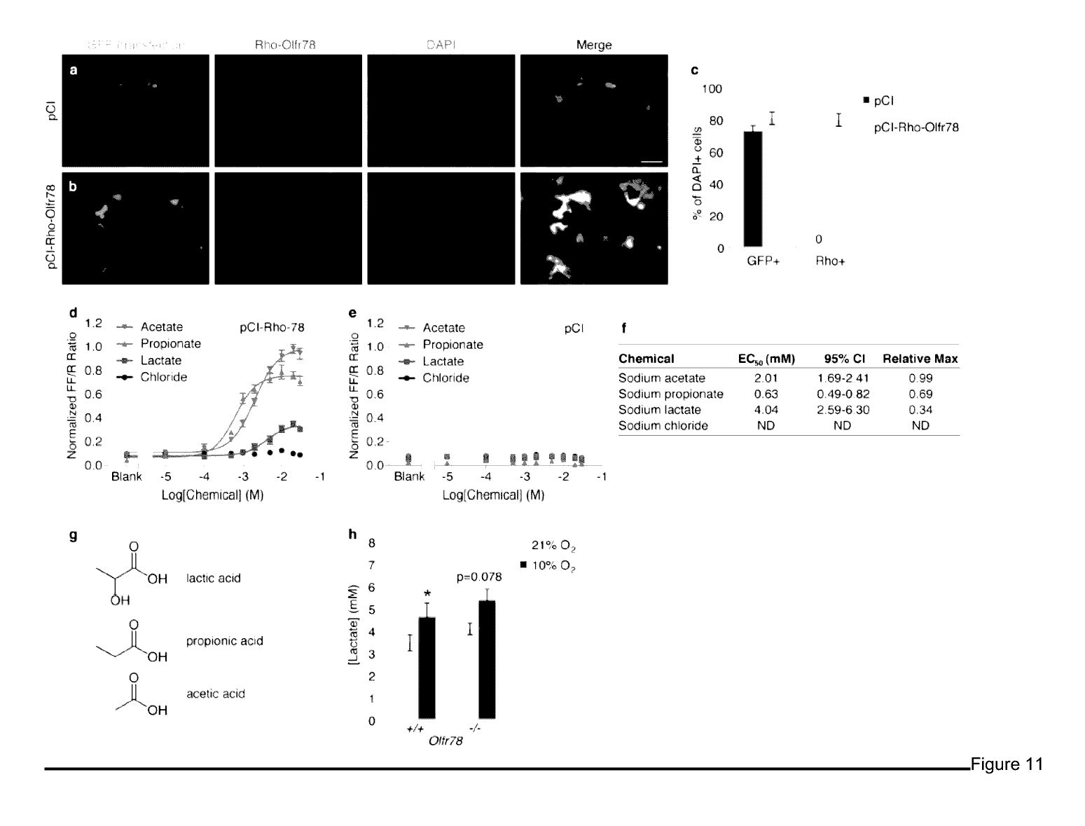

Previous studies showed that mice with fewer carotid body glomus cells have attenuated responses to hypoxia. We examined developmental expression of Olfr78 in carotid body and found it was not expressed embryonically, when transcription factors that regulate carotid body development are detected and glomus cells form. Olfr78 expression was first observed after birth before maturation of carotid body oxygen sensing, and persisted throughout adult life (FIG. 11). The number of TH-positive glomus cells and their organization into clusters were not affected in Olfr78 ⁻⁻ mutants (FIGS. 3a-c). We also did not detect ultrastructural defects: mutant glomus cells still contained the normal large dense core vesicles all along the plasma membrane and small clear core vesicles at synapses² (FIGS. 3d-g). Thus, glomus cells are present in normal numbers and appear structurally intact in Olfr78^{-/-} mutants.

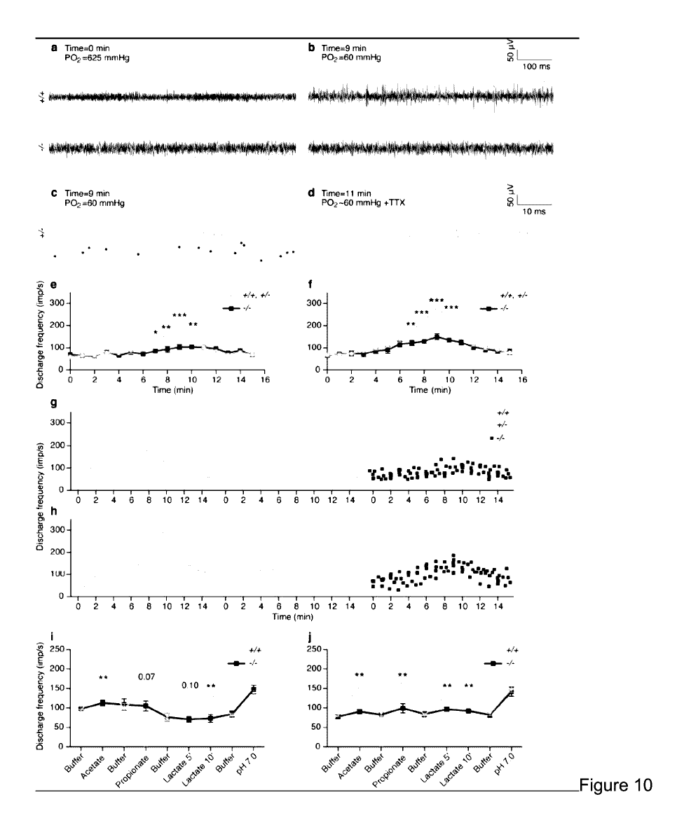

To assess carotid body oxygen sensing, we carried out extracellular recordings of the carotid sinus nerve (FIGS. 5b, c), a standard assay of carotid body neurosensory activity. We found that carotid sinus nerves from Olfr78^{-/-} mutants had similar baseline discharge frequencies as Olfr78^{+/+} and Olfr78^{+/-} controls, demonstrating that nerve activity is intact in Olfr78^{-/-} mutants. However, in hypoxia (PO₂=60-80 mmHg), control nerve activity increased substantially whereas Olfr78^{-/-} mutant nerve activity showed little response (FIG. 3h and FIGS. 10a-h). By contrast, carotid sinus nerve activation by low pH was intact in Olfr78^{-/-} mutants (FIG. 3i and FIGS. 10i, j). We conclude that Olfr78 mutants have a specific defect in carotid body oxygen sensing.

Previous studies demonstrating the robust response of carotid body and breathing to cyanide and other electron transport inhibitors suggest that carotid body oxygen sensing may be mediated by a sensor that detects changes in metabolism (FIGS. 5d, e). Interestingly, two short-chain fatty acids, acetate and propionate, activate Olfr78 and its human ortholog OR51E2 expressed in HEK293T cells, with EC₅₀ values of 1-3 mM. However, blood concentrations of acetate and propionate in rodents and humans are only 0.1-0.3 mM and 4-25 μM, respectively, and change little in hypoxia relative to the Olfr78 activation curve. Thus, we sought a ligand for Olfr78 that is present in blood and tissue and effective at physiologically relevant levels.

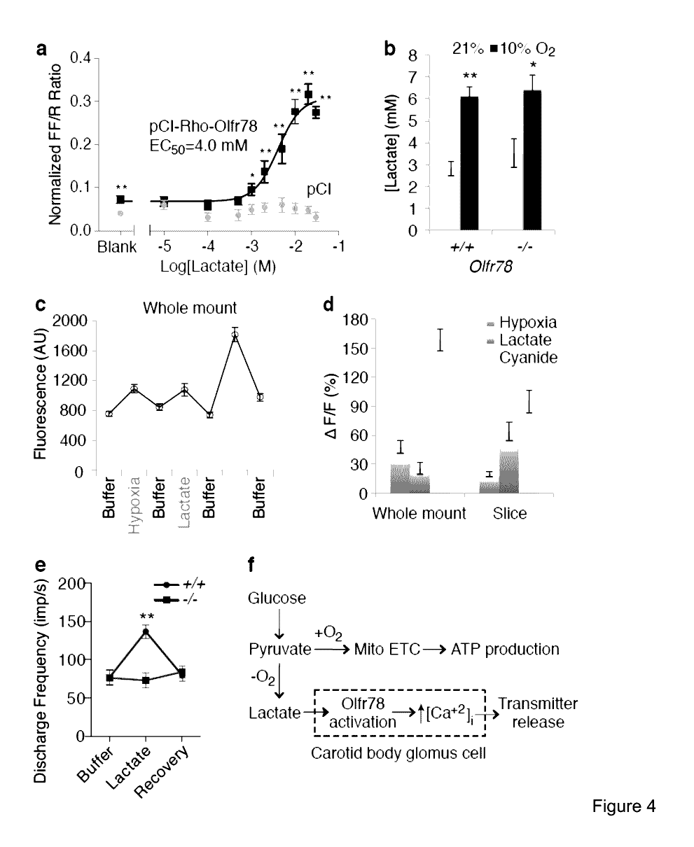

One appealing candidate that is chemically similar to acetate and propionate but more abundant in vivo is lactate, which is found in blood and tissue at low mM concentrations and rapidly increases in hypoxia (FIG. 5e). Using a heterologous expression assay, we found that lactate activated Olfr78 in a dose-dependent manner with an EC₅₀ of 4.0 mM (FIG. 4a and FIGS. 11a-g). Chloride ion over the same range of concentrations and osmolarity had no effect, whereas propionate and acetate stimulated Olfr78 with EC₅₀ values similar to previous findings (FIGS. 11d-g). Because lactate concentrations in blood, tissue, and tissue interstitium are 1-5 mM (FIG. 5e), the observed EC₅₀ value of 4.0 mM for Olfr78 renders it highly sensitive to small changes in lactate in the physiological range. Indeed, hypoxia and mitochondrial poisons such as cyanide elevate plasma and tissue lactate concentrations rapidly in this range (FIG. 5e). We observed that arterial blood lactate increased from 3 mM to 6 mM within 3-5 minutes of hypoxia in both control and Olfr78^{-/-} mutant animals (FIG. 4b and FIG. 11 h). Thus, lactate activates Olfr78 in a physiologically relevant range.

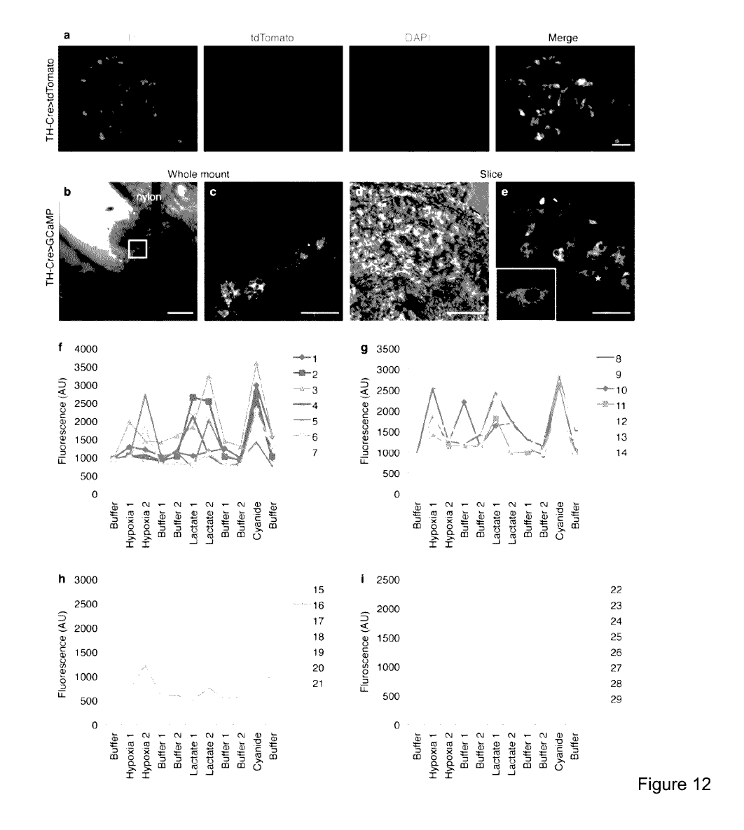

Mitochondrial poisons trigger carotid body glomus cell activity, and acute lactate application depolarizes glomus cells, stimulates carotid sinus nerve activity, and induces hyperventilation. To determine if lactate can directly activate glomus cells, we carried out functional imaging experiments by expressing the calcium indicator GCaMP3 in glomus cells (FIGS. 12a-e). In both whole carotid bodies and slice preparations, we found that lactate induced calcium transients in glomus cells, as did hypoxia or addition of cyanide to inhibit mitochondrial electron transport chain and block oxygen consumption (FIGS. 4c, d and FIGS. 12f-i). The response to lactate was stronger in slices than in intact carotid bodies, perhaps because glomus cells in slices have more direct exposure to lactate in the superperfusate (FIG. 4f). We conclude that lactate can acutely activate glomus cells, much like hypoxia and cyanide. Interestingly, in both these experiments and carotid sinus nerve recordings (see below), carotid body activation by lactate was observed in hyperoxia, suggesting that lactate can stimulate carotid body sensory activity in the absence of other hypoxic signals.

To determine if carotid body activation by lactate requires Olfr78, we examined the effect of lactate on carotid sinus nerve activity in Olfr78^{-/-} mutants. While lactate increased carotid sinus nerve activity in preparations from wild-type Olfr78^{+/+} animals as expected, there was little response to lactate in Olfr78^{-/-} mutant nerves (FIG. 4e and FIGS. 10i, j). Similarly, acetate and propionate, two other Olfr78 ligands that can also stimulate carotid sinus nerve activity, had little effect in Olfr78^{-/-} mutants (FIGS. 10i, j). We conclude that carotid body activation by lactate and two other Olfr78 ligands is mediated by Olfr78.

Our results support a model in which decreased blood oxygen is sensed by the carotid body through an increase in production and secretion of lactate, which binds to Olfr78 on glomus cells and induces calcium transients that increase signaling to afferent nerves to stimulate breathing (FIG. 4f). In the model, changes in blood oxygen are not detected directly by glomus cells, but indirectly through a metabolite (lactate) whose production is regulated by oxygen availability. This explains why drugs and mutations that inhibit the mitochondrial electron transport chain, preventing oxygen utilization and causing lactate buildup, mimic the effect of hypoxia on carotid body activity and breathing, and supports the mitochondrial hypothesis of carotid body oxygen sensing (FIGS. 5d, e). Thus, the Olfr78 pathway measures a metabolic state that integrates oxygen availability and demand and serves as a sentinel that signals, and attempts to stave off, an impending oxygen crisis, whereas the HIF-1 pathway senses oxygen directly (through prolyl hydroxylases that use oxygen to modify HIF-1 stability) and operates later and more broadly to deal with the crisis.

What is the source of the lactate that activates Olfr78? Lactate is produced by all cells in the body when oxygen declines: the blockage of mitochondrial electron transport leads to accumulation of upstream metabolites such as pyruvate, which is rapidly converted to lactate and then effluxed from cells (FIG. 5e). Upon hypoxia exposure, lactate can almost double in blood within minutes (FIG. 4b and FIG. 11h), and it accumulates in blood when inspired oxygen drops to levels that can activate carotid body signaling and hyperventilation. Besides blood, another potential source of lactate is the carotid body itself, as tissue lactate levels also increase rapidly in hypoxia, doubling within 30 seconds in some tissues. Mitochondria of carotid body cells are highly sensitive to hypoxia compared to other tissues (FIG. 5d), so when blood oxygen levels decline, carotid body cells should be among the first to produce lactate, ideal for Olfr78 sentinel function. Because lactate is transported out of cells with H⁺, glomus cells would be exposed to increases in both extracellular lactate and H⁺, which can activate acid-sensitive channels (ASICs, TASKs) synergistically with Olfr78 to stimulate glomus cells. Lactate/Olfr78 signaling may act with H⁺ and perhaps other signals and pathways to promote the full carotid body response to hypoxia, explaining the small residual response to hypoxia detected in Olfr78^{-/-} mutants (FIG. 3h and FIGS. 10a-h).

In addition to the carotid body, Olfr78 is expressed in a number of other oxygen-responsive tissues such as heart and lung, and it is required for maintaining normal blood pressure. We speculate that lactate and Olfr78 serve as a general signal and sensor of hypoxic and altered metabolic states to control physiological responses. Nevertheless, some acute responses to hypoxia, such as reduced locomotion, regular breathing, and metabolic depression, are independent of Olfr78. It may be valuable to develop synthetic agonists and antagonists for Olfr78 for therapeutic control of breathing and other responses it controls.

Genomic studies detect ectopic expression of other ORs in addition to Olfr78 and Olfr558, and some of these appear to be functional. Downstream signaling in the carotid body may differ from that in olfaction (Table 2), and it will be of interest to elucidate the full Olfr78 signal transduction cascade in the carotid body and its integration with other pathways activated by hypoxia and other sensory stimuli. Although olfactory receptors were first identified for their role in smell, they can be involved in myriad chemosensory pathways detecting endogenous and exogenous ligands throughout the body.

Top 150 genes highly expressed in carotid body vs. adrenal medulla by RNA-seq. *CB and AM values are log₂(aligned reads per 10⁷ reads) as FIG. 1a. tLog₂(CB/AM) ratios as in FIG. 6a. All genes were significantly different between CB and AM by paired t test (P<0.05), except Bmx (P=0.056). Yellow highlight, genes for olfactory receptors. Blue highlight, genes previously shown to be expressed in CB. TF, transcription factor; GPCR, G protein-coupled receptor; ROS, reactive oxygen species; CNG, cyclic nucleotide-gated.

### Methods

Animals. All experiments with animals were approved by the Institutional Animal Care and Use Committee (IACUC) at the Stanford University School of Medicine. C57/BL6, Stock #027 (Charles River) was used as wild type for microarrays and RNA sequencing. Other mouse strains used were: Olfr78 knock-in reporter: MOL2.3-IGITL, kindly shared by Ron Yu (Stowers Institute) Olfr78 knock-in mutant/reporter: B6; 129P2-Olfr78^{tm1Mom}/MomJ, Stock #006722 (JAX)^{;} Olfr558 mutant/reporter: B6129S5-Olfr558^{tm1Lex}, Stock #TF0586 (Taconic); Th-Cre driver: B6.FVB(Cg)-Tg(Th-cre)^{FI172Gsat}/Mmucd, Stock #031029-UCD (MMRRC) Th-Cre driver: Th^{tm1(cre)Te}, kindly shared by Karl Deisseroth (Stanford)^{;} ROSA-GCaMP3: B6;129S-Gt(ROSA)26Sor^{tm38(CAG-GCaMP3)Hze}/J, Stock #014538 (JAX)^{;} ROSA-tdTomato: B6;129S6-Gt(ROSA)26Sor^{tm9(CAG-tdTomato)Hze}/J, Stock #007905 (JAX)^{.}

Adult animals were used in all experiments, unless indicated otherwise. To control for sex differences, only female animals were used in physiology and behavioral experiments. Randomization and blinding were not used, in part because the Olfr78 mutant allele is genetically linked to a coat color variant. All data include animals from multiple litters.

Carotid body and adrenal medulla RNA purification. Adult C57/BL6 animals were anesthetized with isoflurane and decapitated, and carotid bifurcations and adrenal glands were dissected immediately and transferred to RNA/ater solution (Life Technologies) on ice. Carotid bodies and adrenal medullas were finely dissected from these tissues. From each animal, 1-2 carotid bodies and 1 adrenal medulla were obtained and stored in RNA/ater at 4° C. for up to 2 days. For each RNA purification, 18 carotid bodies and 10 adrenal medullas from 10 animals were pooled and processed using the RNeasy Micro Kit (Qiagen). Tissue pieces were disrupted in a guanidine-isothiocyanate lysis buffer (Buffer RLT, Qiagen) using a glass tissue grinder (Corning), homogenized using a 20 G needle and syringe, and purified by silica-membrane columns. RNA quality was assessed by electrophoresis on a Bioanalyzer using the RNA 6000 Pico Kit (Agilent). The average RNA Integrity Numbers (RIN) for carotid body and adrenal medulla samples were 7.2 and 9.0, respectively.

Microarrays. Total RNA (>30 ng/sample) was processed using the 3′ IVT Express Kit (Affymetrix) to make biotinylated amplified RNA (aRNA) by cDNA synthesis and in vitro transcription. aRNA was fragmented and hybridized to the GeneChip Mouse Genome 430 2.0 Array (Affymetrix) containing 45,000 probe sets targeting >34,000 mouse genes. aRNA synthesis, hybridization, and scanning were performed by the Stanford PAN Facility. Analysis of microarray data was performed using Expression Console and Transcriptome Analysis Console software (Affymetrix).

RNA sequencing. Using the Amino Allyl MessageAmp II aRNA Amplification Kit (Ambion), unlabeled aRNA was generated from total RNA (30-50 ng/sample) by an engineered M-MLV reverse transcriptase to make cDNA followed by in vitro transcription. aRNA was fragmented by RNA Fragmentation Reagents (Ambion), a buffered zinc solution, for 1.5 minutes at 70° C. First and second strand cDNA synthesis, end repair, 3′-dA tail addition, and adaptor ligation were performed using the standard protocol from Illumina with adaptor oligonucleotides from Illumina, First Strand Buffer, SuperScript III reverse transcriptase, and Second Strand Buffer from Invitrogen, and RNaseH, DNA polymerase I, T4 DNA polymerase, Klenow DNA polymerase, T4 polynucleotide kinase, Klenow fragment (3′-5′ exo-), and T4 DNA ligase from New England Biolabs. Modified cDNA libraries were resolved by electrophoresis in 2% low-melting agarose (Lonza) gels. For each sample, a region of the lane corresponding to —200 base pairs was excised and purified by the QlAquick Gel Extraction Kit (Qiagen) using silica-membrane columns. Modified cDNA libraries were further amplified by PCR for 20 cycles using Phusion DNA polymerase (New England Biolabs). cDNA concentration and size were determined by electrophoresis using the High Sensitivity DNA Kit on the Bioanalyzer (Agilent), and samples were diluted to 4 pM for sequencing.

DNA clusters were generated using the Cluster Generation Kit according to manufacturer instructions (Illumina). Samples were then sequenced on the Illumina Genome Analyzer II using the 36-Cycle SBS Reagent Kit v2 (Illumina) run for 38 cycles. Each cDNA library was run in one lane, and data presented in this study are from two separate runs.

Sequences were aligned to the RefSeq database using Bowtie 0.9.8, allowing up to 4 mismatches in the first 32 bases for a sequence to be assigned to a specific gene ID. Reads that mapped to multiple isoforms of a gene were randomly assigned to one isoform, and counts for multiple mRNA isoforms for the same gene were combined for analysis. The number of aligned reads per 10⁷ aligned reads was calculated after adding 1 read to every gene and sample in order to avoid dividing by 0 when calculating ratios between AM and CB frequencies (FIG. 1a and FIG. 6a). Reads per kilobase per million (RPKM) values were calculated by using the length of the mRNA, or the longest mRNA isoform for genes that have multiple isoforms, in RefSeq (FIG. 1 b and FIG. 6b).

X-gal staining. Whole mount carotid bifurcations and adrenal glands were harvested, cleaned, and fixed in 4% paraformaldehyde (PFA)/PBS (pH 7.4) for 10 minutes at room temperature. After washing with PBS, tissue was transferred to a solution of X-gal (1 mg/ml), potassium ferricyanide (5 mM), potassium ferrocyanide (5 mM), magnesium chloride (2 mM), and NP-40 (0.02%) in PBS and incubated overnight at 37° C. Samples were visualized on a Leica MZ12 stereomicroscope. Representative data reflect tissue samples from ten Olfr78-GFP-taulacZ^{Tg/Tg} (FIGS. 1c-f, i-k) and three Olfr558^{1acZ/tacZ} (FIG. 7e) animals.

For carotid bifurcation samples shown in section (n=3 animals), tissue was fixed in 4% PFA/PBS for 10 minutes at room temperature and equilibrated in 30% sucrose overnight at 4° C. Tissue was then embedded in optimum cutting temperature compound (O.C.T., TissueTek) and stored at −80° C. Sections were cut at 10 μm using a Leica CM3050S cryostat. X-gal solution was added onto sections on slides and incubated overnight at 37° C. Slides were mounted in Mowiol 4-88 (Polysciences) with 1,4-diazabicyclo[2.2.2]octane (DABCO, 25 mg/ml, Sigma-Aldrich) or Permount (45% polymer of α-pinene, β-pinene, dipentene, β-phellandrene/55% toluene, Fisher) and visualized on a Zeiss Axiophot fluorescence microscope.

For adult brain tissue, two animals were perfused through the heart with PBS followed by 4% PFA/PBS using a syringe. Whole brains were dissected from the head, and fixed again for 30 minutes in 4% PFA/PBS at 4° C. After equilibration in 30% sucrose overnight at 4° C., samples were embedded in O.C.T. (TissueTek) and sectioned at 80 μm using a Leica CM3050S cryostat. Slides were then incubated with X-gal overnight at room temperature and visualized on a Leica MZ12 stereomicroscope.

Immunostaining. Tissue was fixed in 4% PFA/PBS at room temperature for 10 minutes and equilibrated in 30% sucrose overnight at 4° C. Tissue was embedded in O.C.T. (TissueTek) and stored at −80° C. Sections were cut at 10 μm using a Leica CM3050S cryostat and incubated with primary antibodies overnight at 4° C. Primary antibodies were rabbit anti-TH (Abcam, ab112), chicken anti-GFP (Abcam, ab13970), rat anti-CD31 (BD Pharmingen, 553370), chicken anti-β-galactosidase (Abcam, ab9361), and mouse anti-smooth muscle actin (Sigma, A5228) used at 1:500. Mouse anti-smooth muscle actin antibody was directly conjugated to Cy5 NHS ester (GE Healthcare), and unbound dye was removed on a P-30 gel exclusion column (BioRad). Incubation with secondary antibodies was 45 minutes at room temperature. Secondary antibodies were conjugated to either Alexa Fluor 488, Alexa Fluor 555 (Life Technologies), or DyLight 488 (Jackson ImmunoResearch). Staining with 4′,6-diamidino-2-phenylindole (DAPI; 1 ng/ml, Life Technologies) was performed after incubation with secondary antibodies for 5 minutes at room temperature. Sections were mounted in Mowiol 4-88 (Polysciences) with DABCO (25 mg/ml, Sigma-Aldrich) and visualized on a Zeiss Axiophot fluorescence microscope. Tissue samples from three or more animals were stained for representative data shown (FIGS. 1g, h, FIGS. 3a, b, and FIGS. 7a-c, f-h).

Electron microscopy. Carotid bifurcations were harvested from adult animals and transferred to fixation solution (4% PFA and 2% glutaraldehyde in PBS) for 1 hour at room temperature. During fixation, excess tissue was trimmed away to retain the carotid body and carotid arteries. Samples were post-fixed with osmium tetroxide for 1.75 hours at 4° C., washed three times with cold double distilled H₂O, and incubated with 1% uranyl acetate overnight at 4° C. On the next day, samples were serially dehydrated in ethanol (50%, 70%, 100%, 100%) for 10 minutes per step and washed with propylene oxide for 15 minutes, all at room temperature. Samples were then transferred to 1:1 propylene oxide: Epon (Electron Microscopy Services) for 1 hour, 1:2 propylene oxide: Epon for 45 minutes, and 100% Epon, all at room temperature. Once samples were embedded in 100% Epon, blocks were baked overnight at 65° C.

To locate the carotid body in the embedded tissue, sections were cut at 2 μm using glass knives on a Leica Ultracut S microtome and stained with Toluidine Blue (Sigma-Aldrich) for visualization of tissue histology. Once the carotid body was reached, 17 nm sections were cut using a diamond blade for transmission electron microscopy. Sections were visualized on a JEOL TEM1230 transmission electron microscope equipped with a Gatan 967 slow-scan, cooled CCD camera. Two sections at different levels in the carotid body were examined for each sample. All sectioning and imaging procedures were performed at the Stanford Cell Sciences Imaging Facility-Electron Microscopy Core.

Whole body plethysmography. Unrestrained, unanesthetized adult animals were transferred to a whole body plethysmograph (450 ml, Model PY4211, Buxco) connected to a MAXII preamplifier unit and computer running BioSystem XA software (Buxco). To reach stable baseline ventilation, animals were acclimatized to the chamber for more than 30 minutes in control gas conditions before exposure to hypoxia or hypercapnia. Three pulses of hypoxia or hypercapnia lasting 5 minutes each were performed with 10-minute recovery periods in control conditions. Gas mixtures for control, hypoxia, and hypercapnia were 21% O₂/79% N₂, 10% O₂/90% N₂, and 5% CO₂/21% O₂/74% N₂, respectively (Praxair). Flow rates were 1.5 L/min during measurement periods and 11-12 L/min during 1-minute ramp periods between gas mixtures.

Ventilatory parameters were collected and calculated by BioSystem XA software (Buxco). Tidal volumes were calculated according to Drorbaugh and Fenn with manual input of environmental conditions, such as room and chamber temperature, humidity, and barometric pressure. To enrich for measurements of regular breaths, criteria were set in the software to accept a breath if (1) inspiratory time was greater than 0.07 seconds, (2) expiratory time was less than 10 seconds, (3) calculated tidal volume was greater than 0.05 ml, and (4) volume balance between inspiration and expiration was less than 50%. Under these conditions, virtually all breaths were accepted during very regular breathing in hypoxia and hypercapnia. Fifteen breaths were averaged for each line of data, and lines of data for each period of control or stimulus were averaged, excluding lines that had more than two observed events of sniffing, grooming, or movement among the accepted breaths. Ventilatory parameters over all periods of control or stimulus were averaged for each animal and presented in the figures. Most animals were tested two times within one week with good reproducibility, and measurements were averaged. Numbers of animals tested were comparable to other published work³⁷⁻⁴⁰. While there was no formal randomization, different numbers, genotypes, and litters were tested in different orders on multiple days over several months. For the animals used in our study, body weight did not correlate with respiratory rate, tidal volume, or minute ventilation in wild-type animals, mutant animals, or all animals together (correlation coefficients 0.001≦R²≦0.289, 0.996≧P≧0.293).

Blood gas and lactate measurements. For blood testing under anesthesized conditions, animals were transferred to individual cages the morning of testing and allowed to acclimate for at least 4 hours. This was designed to avoid repeated cage handling and removal of other animals from the same cage, procedures that have been shown to increase stress hormones and blood lactate levels in rodents. Animals were quickly anesthesized with 3% isoflurane in 100% O₂ in an acrylic container and maintained in 1.5-2% isoflurane in 21% O₂/79% N₂ or 10% O₂/90% N₂ (Praxair) at 2 L/min through a nose cone. Body temperature was maintained at 37° C. using a heating pad with feedback temperature controller (Physitemp Instruments). The right carotid artery was surgically isolated and cut, and 200-250 μl of blood was collected using a heparinized syringe. An aliquot (˜100 μl) of arterial blood was immediately transferred to a CG4+ cartridge for measurement of blood gases and lactate using an i-Stat Portable Clinical Analyzer (Abbott). Time from beginning of surgery to blood collection was ˜3 min, during which the hypoxic ventilatory response was still robust under our conditions. For some animals, arterial blood was also transferred to a test strip for lactate analysis using a Lactate Scout analyzer (EKF Diagnostics). We found good correlation between lactate measurements of the same blood sample from anesthesized animals using i-Stat and Lactate Scout analyzers (n=11, R²=0.92, P<0.0001).

For blood lactate measurements in unanesthetized conditions, animals were transferred to individual cages at least one day before the first day of testing. Animals in their housing cage were moved into a hypoxia control glove box set to 21% O₂ or 10% O₂ balanced by N₂ (Coy Laboratory Products). After 1 min in the airlock, the cage was moved into the glove box, and the lid was opened for another 3 min. Then the animal was transferred to a tail vein restrainer (Braintree Scientific), and the tail artery was punctured with a 27 G% needle. Blood was then directly transferred to a test strip for measurement of lactate using a Lactate Scout analyzer. The total time of animals in the glove box before blood testing was 4-5 min. Due to handling stress increasing blood lactate concentrations and causing more variable blood lactate measurements in awake conditions, animals were kept in the same room and tested on 4 separate days for 2 days each of 21% O₂ or 10% O₂ exposure. Results for each animal and oxygen condition were then averaged. One Olfr78^{+/+} animal was excluded because of excessive handling stress due to long blood collection on 2 days.

Body temperature measurements. Unanesthetized animals were transferred to a tail vein restrainer (Braintree Scientific), and body temperature was measured using a rectal temperature probe and animal temperature controller (Physitemp Instruments) in room air (21% O₂) or in 10% O₂ in a hypoxia control glove box (Coy Laboratory Products). Data were collected in 21% O₂ at 1 hour before transfer to the hypoxia control glove box and at 2 and 5 minutes in 10% O₂ inside the glove box. An airlock was used to transfer the animal into the glove box for a ramp time of 1 minute from 21% O₂ to 10% O₂.

Metabolic measurements. Unrestrained, unanesthetized animals were transferred to the same chamber used for plethysmography that was sealed to only allow airflow in from the side port and out from a bottom port on the opposite side. Metabolic measurements were collected using an Oxymax open circuit indirect calorimeter (Columbus Instruments) with an electrochemical oxygen sensor modified to measure two ranges around 21% O₂ and 10% O₂. Flow was set to 0.6 L/min from gas mixtures of 21% O₂/79% N₂ and 10% O₂/90% N₂ (Praxair). Measurements were taken every 30 seconds. For 21% O₂, animals were allowed to acclimate to the chamber for 10 minutes, and data is shown for 10-15 minutes after the start of measurements. For 10% O₂, animals became calm more quickly, and data is shown for 5-10 minutes after the start of measurements, a duration we found necessary for the system to stabilize to 10% O₂ after opening the chamber.

Measurements of oxygen in perfusion. Measurements of oxygen concentrations of the perfusion solution in the recording chamber were performed using a Clark style oxygen electrode (Unisense). Because voltage readings for the sensor were observed to be highly temperature-dependent, the sensor was calibrated at the temperature of the relevant protocol, which was 33-34° C. for electrophysiology and room temperature for calcium imaging.

Carotid sinus nerve recordings. Animals were terminally anesthetized with isoflurane, perfused through the heart with ice-cold artificial cerebrospinal fluid (ACSF, pH 7.4) composed of 119 mM NaCl, 5 mM KCl, 2.5 mM CaCl₂, 1.3 mM MgSO₄, 1 mM NaH₂PO₄, 26.2 mM NaHCO₃, and 11 mM glucose previously bubbled with 95% O₂/5% CO₂ (Praxair), and decapitated. Both carotid bifurcations were then dissected in ice-cold ACSF and transferred to a recording chamber (3 ml), where they were superperfused with ACSF continuously bubbled with 95% O₂/5% CO₂ (Praxair) at a flow rate of 13.3 ml/min by gravity and maintained at 33-34° C. The carotid sinus nerve was carefully exposed and cut near the point where it branches from the glossopharyngeal nerve. The cut end was pulled into a tightly fitting glass suction micropipette, and voltage was recorded relative to a reference in the bath using an Axoclamp 2A electrometer (Molecular Devices) in Bridge mode. The voltage signal was amplified 1000× (10× on the Axoclamp and 100× on a Brownlee Precision Model 440 instrumentation amplifier), filtered (0.2-3 kHz), and digitized at 10 kHz on a National Instruments MIO15E-2 analog-to-digital converter. Data were stored and displayed using in house software written in LabView (National Instruments).

If spikes were not observed at baseline for the first carotid sinus nerve, the second carotid bifurcation was dissected and recorded. One-second sweeps were acquired continuously through the entire time course, and only one carotid sinus nerve recording per animal was included in the data presented. Hypoxia stimulus was delivered by changing the gas bubbling the ACSF to 95% N₂/5% CO₂ (Praxair) for 8 min. Under these conditions, PO₂ levels in the recording chamber started at 625 mmHg and decreased to a low of 60 mmHg by 9 min after the start of bubbling with 95% N₂/5% CO₂ (FIG. 3h). Solutions of lactate, acetate, and propionate (30 mM, pH 7.4) were made by equimolar substitution of NaCl in ACSF with sodium salts of L-lactate, acetate, and propionate, respectively. Low pH solution (pH 7.0) was made by lowering the NaHCO₃ in ACSF from 26.2 mM to 11.9 mM with an equimolar increase in NaCl³⁸. These solutions were continuously bubbled with 95% O₂/5% CO₂ (Praxair) at the reservoir, maintaining the oxygen concentration of the solution in the chamber at PO₂=625 mmHg. All preparations were stimulated with bolus injections of 25-50 μl sodium cyanide (20 mM) at the end of the experiment to confirm that the nerve was active.

Recordings were analyzed offline using Spike2 software (Cambridge Electronic Design). To measure action potential frequency, we analyzed a one-second period of data every minute. A single threshold was used for spike determination and set empirically for each time course by moving the threshold through a range of values until the spike count stabilized through several intervals of 0.001 mV and then dropped off for data at time=0 and 9 min (hypoxia) or all time points scored (acetate, propionate, lactate, and low pH). The lowest threshold value in the stable range was applied to all sweeps of a stimulus analyzed for each recording. Because we noticed that spikes close together or with low amplitudes were often missed by the software, we also manually counted spikes for the same sweeps analyzed by software. Two recordings were excluded due to low signal to noise precluding accurate analysis. In two experiments, we also applied 7.5 μM tetrodotoxin (TTX) to block voltage-gated sodium ion channels during hypoxia exposure as an additional confirmation that events being scored were action potentials (FIGS. 10c, d).

pCl-Rho-Olfr78 plasmid construction and expression. The pCl-Rho-Olfr78 plasmid was made by PCR amplifying the Olfr78 coding region from a pCMV6-Olfr78 plasmid (OriGene) using forward primer 5′-ATTGCCGAATTCATGAGTTCCTGCAACTTCACC-3′ and reverse primer 5′-ATTGCCGCGGCCGCTCACGTGTTTCCCCCAGCTTCAA-3′, adding EcoR1 and Not1 restriction sites. The Olfr78 PCR fragment was then digested with EcoR1 and Not1 and cloned into a pCl-Rho backbone cut from a pCl-Rho-Olfr62 plasmid (gift of Hiroaki Matsunami, Duke University). Cell-surface expression of Rho-epitope-tagged Olfr78 protein in HEK293T cells was confirmed by immunostaining. Cytoplasmic GFP (co-transfection marker) was expressed from a TBC1 D25::eGFP plasmid (gift of Suzanne Pfeffer, Stanford University).

Luciferase assay. HEK293T cells were grown and seeded into 96 well plates. On the next day, cells were transfected with RTP1S, G_{α15-olf} (gifts of Hiroaki Matsunami, Duke University), pCMV6-Ric8b (Origene), pCRE-Luc (Agilent), and pSV40-RL (Promega) plasmids and either pCl-Rho-Olfr78 or pCl (Promega). The Rho tag on Olfr78 and RTP1S were used to enhance localization of Olfr78 to the plasma membrane. G_{α15-olf} and Ric8b were included as downstream effectors that couple to ORs to increase cAMP production upon OR activation. Two transcriptional luciferase reporters, one constitutive (Renilla, pSV40-RL) and one inducible by cAMP (firefly, pCRE-Luc) were transfected to report increased cAMP levels upon OR activation.

Five hours after transfection, media was decanted and replaced with 50 μL/well MEM without phenol red (Life Technologies). After thirty minutes, 25 μL of chemicals were added to each well to achieve the indicated final concentrations, and cells were incubated for 2 hours. Sodium salts of chloride, L-lactate, propionate, and acetate (Sigma) were used. The duration of transfection and stimulation was shortened because we observed that transfection of cells with pCl-Rho-Olfr78 overnight caused a large increase in firefly luciferase activity in the absence of added chemicals, suggesting that lactate or some other molecule released from cells and/or a component of the transfection mixture could stimulate Olfr78 activity.

Reagents to detect firefly and Renilla luciferase activity (Dual-Glo Luciferase Assay System, Promega) were added at 20 μl/well. Luminescence was measured using an Infinite M1000 (Tecan) microplate reader and data acquired by Magellan Data Analysis Software (Tecan). Two readings were collected for each plate and luciferase reagent, and firefly to Renilla ratios were averaged. Data was from experiments conducted over three days. For dose response curves in FIG. 4a and FIGS. 11d-f, ratios were normalized to the highest and lowest average values for a given condition across all plates on each day.

The HEK293T cell line was not authenticated or tested for mycoplasma contamination.

Calcium imaging. Th-Cre; ROSH-GCaMP3 animals expressing GCaMP3 in glomus cells were generated using two Th-Cre drivers. Both Th-Cre lines drove expression in glomus cells, as confirmed by Th-Cre; ROSA-tdTomato animals, but the BAC-transgenic Th-Cre driver (MMRRC) required two copies of ROSA-GCaMP3 reporter for robust expression. For whole mount preparations, carotid bifurcations were dissected from transgenic animals and transferred to 0.5% glucose/PBS bubbling 100% O₂ on ice. Surrounding tissue was removed to expose the carotid body attached to the carotid artery. The carotid body was incubated in a physiological buffer (115 mM NaCl, 5 mM KCl, 24 mM NaHCO₃, 1 mM MgCl₂, 2 mM CaCl₂, 11 mM glucose) at 37° C. in a tissue culture incubator with 5% CO₂ before transfer to the recording chamber for imaging.

For tissue slices, carotid bifurcations were dissected and transferred to a modified Tyrode's solution (148 mM NaCl, 2 mM KCl, 3 mM MgCl₂, 10 mM HEPES, 10 mM glucose), pH 7.4 on ice. Carotid bodies were then isolated and embedded in 3% low melt agarose (Lonza) in a sample holder (Precisionary Instruments). Tissue slices were cut at 100 μm using a Compresstome VF-200 (Precisionary Instruments). Samples were then transferred to culture medium composed of DMEM with 10% FBS, 1% penicillin/streptomycin, and insulin-transferrin-selenium (Life Technologies) and incubated in a tissue culture incubator at 37° C. with 5% CO₂ for at least 24 hours before calcium imaging according to established protocols.

At baseline, the carotid body was superperfused with physiological buffer bubbling 95% O₂/5% CO₂ at 3.75 ml/min using a Reglo analog tubing pump (Ismatec), maintaining the oxygen concentration of the solution in the chamber at PO₂=600 mmHg. Hypoxia was generated by bubbling physiological buffer with 95% N₂/5% CO₂. Lactate solution (30 mM) was made by equimolar substitution of NaCl with sodium L-lactate. Lactate and cyanide solutions were bubbled with 95% O₂/5% CO₂. To switch between stimuli, the flow rate was increased to 7.5 ml/min for 2 minutes.

The carotid body was imaged on a Prairie Ultima XY two-photon rig built around an Olympus BX-61W upright microscope at the Stanford Neuroscience Microscopy Service. Using a water immersion 60× objective, Z-stacks at 2 μm steps were collected at 1024×1024 pixels resolution for 70-100 μm of tissue. Two stacks were collected for hypoxia and lactate stimuli and intervening buffer recovery periods for whole mount samples. Images were analyzed using ImageJ. Regions of interest corresponding to individual glomus cells were defined by the images with the strongest fluorescence. Average fluorescence intensities were calculated for each region of interest, and values were averaged for stimulus and buffer periods with more than one stack. Cells that had very high levels of fluorescence at the start of the experiment were excluded from our analysis of data from whole mount samples because these cells showed dramatic declines in baseline fluorescence after hypoxia and lactate stimulation. Data presented are from 2 samples from 2 different animals performed on separate days.

Data analysis and statistics. Data analysis and statistical tests were performed using Microsoft Excel and GraphPad software. GraphPad Prism 6 was used to fit dose-response curves using a variable slope model and to calculate EC₅₀ values. All data are biological replicates, and quantitative data with error bars are presented as mean±standard error of the mean (s.e.m.) with the exception of FIG. 11c, which is presented as percent±standard error of percentage. Groups compared by parametric tests fit the assumption for normal distribution as determined by the Shapiro-Wilk test with the critical W value set at 5% significance level. All t tests shown are two-sided, and variances of sample groups compared were similar. No statistical method was used to predetermine sample size.

What is claimed is:

1. A method of modulating hypoxia-regulated breathing in a mammal through inhibiting or activating the chemosensory receptor Olfr78/OR51E2, the method comprising:
contacting cells of the carotid body in a mammalian subject with an effective of a ligand of Olfr78/OR51E2.

2. The method of claim 1, wherein the ligand is an agonist.

3. The method of claim 2, wherein the effective dose is sufficient to increase ventilation by at least about 25%.

4. The method of claim 3, wherein the subject is an infant.

5. The method of claim 3, wherein the subject suffers from sleep disordered breathing.

6. The method of claim 3, wherein the subject is treated with an opiate or volatile anesthetic that induces breathing depression.

7. The method of claim 1, wherein the ligand is an antagonist.

8. The method of claim 7, wherein the effective dose decreases hyperstimulation of the carotid body.

9. The method of claim 8, wherein the subject suffers from congestive heart failure.

10. The method of claim 8, wherein the subject suffers from hypertension.

11. The method of claim 9, wherein the effective dose is sufficient to decrease blood pressure of the subject.

12. A composition for use in the method of any one of claim 1.

13. The composition of claim 12, comprising an effective dose of a ligand of Olfr78/OR51E2, and a pharmaceutically acceptable carrier.

14. The composition of claim 13, wherein the ligand is identified by the method of contacting Olfr78/OR51E2 with a candidate ligand, and determining the effect of the candidate ligand on receptor activation.

15. The method of claim 14, wherein the receptor is present on the surface of a cell.

16. The method of claim 14, wherein a candidate ligand is further screened for activity regulating ventilation in vivo.
